(19) United States
(12) Patent Application Publication
(10) Pub. No.: US 2004/0005358 A1
(43) Pub. Date: Jan. 8, 2004

(54) Modified-release vasopeptidase inhibitor formulation, combinations and method

(76) Inventors: Peter H. Slugg, Princeton, NJ (US); Nemichand B. Jain, West Windsor, NJ (US); Rajesh Krishna, West Windsor, NJ (US); Robert L. Jerzewski, Belle Mead, NJ (US); Ronald L. Smith, Yardley, PA (US); Jatin M. Patel, West Windsor, NJ (US); Bimal K. Malhotra, New Brunswick, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/419,397

(22) Filed: Apr. 21, 2003

Related U.S. Application Data
(60) Provisional application No. 60/374,940, filed on Apr. 23, 2002.

(51) Int. Cl. A61K 31/554; A61K 9/22

(52) U.S. Cl. 424/468; 514/211.05

(57) ABSTRACT

A modified-release vasopeptidase inhibitor formulation is provided which is capable of releasing vasopeptidase inhibitor, preferably omapatrilat or gemopatrilat, in a manner to provide therapeutically effective NEP inhibitory activity and therapeutically effective ACE inhibitory activity, in a balanced manner, for a predetermined duration to lower blood pressure and/or treat heart failure. In a preferred formulation, the NEP inhibitory activity and ACE inhibitory activity have balanced release characteristics.

Combinations including the above formulation and a diuretic and/or other therapeutic agents and methods for lowering blood pressure employing the above formulation and/or combination and a method for enhancing or balancing NEP inhibitory activity of a vasopeptidase inhibitor are also provided.

### FIELD OF THE INVENTION

This application claims a benefit of priority from U.S. Provisional Application No. 60/374,940, filed on Apr. 23, 2002, the entire disclosure of which is herein incorporated by reference.

The present invention relates to a modified-release vasopeptidase inhibitor formulation for use in lowering blood pressure and treating congestive heart failure and other cardiovascular diseases, which formulation provides for balanced NEP inhibitor activity (as compared to comparable rapid-release formulations) without adversely affecting ACE activity, to pharmaceutical combinations including such modified-release formulation and other therapeutic agents including diuretics, and to method for reducing blood pressure and/or treating congestive heart failure employing such modified-release formulation.

### BACKGROUND OF THE INVENTION

Controlled (or modified)-release drug delivery devices have been employed to deliver drug to the patient over a desired period of time. These devices may be characterized as diffusion controlled systems, osmotic dispensing devices, dissolution controlled matrices, or erodible/degradable matrices. Examples of such devices are disclosed in U.S. Pat. No. 3,538,214 (diffusion controlled device); U.S. Pat. Nos. 3,845,770, and 3,916,899, 4,526,108 and 4,612,008 (osmotic devices); Controlled Drug Delivery: Fundamentals and Applications, 2 Ed., J. R. Robinson et al (1987), and Controlled Drug Delivery: Basic Concepts, Vols. I and II, S. D. Brunk, Ed. (1983) (diffusion controlled and erodible/degradable devices).

It is also known that modified-release drug delivery enhances the therapeutic efficiency and pharmacodynamic availability of members of various classes of drugs including corticosteroids, diuretics, growth hormone, erythropoietin and insulin, “Role of dosage regimen in controlling indirect pharmacodynamic responses”, J. V. Gobburu et al, Advanced Drug Delivery Reviews 46 (2001), 45-57.

Modified-release coatings have long been used to release drug from tablets and beads at desired sites of absorption. These coatings, depending on the composition and/or thickness thereof, dissolve and allow for the release of drug in not only the stomach but in the small intestine as well. Some examples of coatings previously employed are beeswax and glyceryl monostearate; beeswax, shellac and cellulose; and cetyl alcohol, mastic and shellac as well as shellac and stearic acid (U.S. Pat. No. 2,809,918); polyvinylacetate and ethyl cellulose (U.S. Pat. No. 3,835,221); neutral copolymer of polymethacrylic acid esters (Eudragit L30D) (F. W. Goodhart et al, Pharm. Tech., pp 64-71, Apr. 1984); copolymers of methacrylic acid and methacrylic acid methyl ester (Eudragits) or a neutral copolymer of polymethacrylic acid esters containing metallic stearates (U.S. Pat. Nos. 4,728,512 and 4,794,001 to Mehta et al.); hydroxypropylmethyl cellulose phthalate (HPMCP) (Shin-Etsu Chemical Co., Ltd.)(U.S. Pat. No. 5,225,202).

EP 1,063,973 discloses a matrix tablet especially designed for highly soluble drugs such as metformin, which matrix tablet includes (1) an inner solid particulate phase in the form of individual granules or particles containing a drug which has a high water solubility, and an extended release material, such as various hydrophobic polymers, and (2) an outer solid continuous phase in which granules or particles of inner solid particulate phase are dispersed and embedded, the outer solid continuous phase being formed of an extended release material, such as various hydrophilic polymers and/or hydrophobic polymers.

Pharmaceutical compositions which include a medicament which is absorbed in the stomach, small intestine and large intestine will require a modified-release coating or matrix to allow for release of such medicament in both the stomach and small intestine, and the colon.

Until now, controlled- or modified-release drug delivery systems including matrix systems have not been employed to deliver vasopeptidase inhibitors.

Vasopeptidase inhibitors (also referred to as NEP/ACE inhibitors, neutral metalloendopeptidase inhibitors or dual action inhibitors) are a new class of drugs that possess dual mechanism of action. Agents in this class inhibit both neutral endopeptidase (NEP) and angiotensin converting enzyme (ACE). Inhibition of neutral endopeptidase prevents the breakdown of several endogenous vasodilator peptides. Inhibition of ACE decreases the formation of the vasoconstrictor peptide angiotensin II. In addition, inhibition of both enzymes reduces the degradation of the vasodilator peptide bradykinin.

ACE inhibitors (captopril, enalapril maleate, lisinopril, and the like) have been used to treat hypertension and congestive heart failure since the 1980's. NEP inhibitors have also been investigated for utility in cardiovascular diseases.

Vasopeptidase inhibitors have been used for the treatment of hypertension and several major sequelae, including left ventricular hypertrophy, congestive heart failure, angina and cardiovascular morbidity and mortality.

For some agents within the class of vasopeptidase inhibitors, the inhibition profiles for ACE and NEP are dissimilar in that ACE inhibition tends to have a longer more balanced duration than NEP inhibition for a given dose. For example, a dose of a vasopeptidase inhibitor may inhibit ACE (in a substantially balanced manner) over a 24 hour period whereas the level of NEP inhibition from the same dose may surge or peak during the first few hours of release (as compared to the more balanced level of ACE inhibition during such period of release) and then taper off during the remainder of its duration. The surge in the level of NEP inhibitory activity may be undesirable since it could lead to a rapid increase in levels of vasodilator peptides and may result in adverse events such as flushing, angioedema and hypotension. Thus, the benefits of such dual action(ACE/NEP) inhibitors could be further optimized if the NEP inhibition profile could be modified and balanced independently from the already satisfactory ACE inhibition profile.

It is known that some agents in this class produce less than optimal trough-to-peak ratios for the blood pressure lowering effects, resulting in a variance of blood pressure over the dosing time.

Inasmuch as vasopeptidase inhibitors represent a valuable new class of therapeutic agents, improved dosage forms which provide for improved balanced activity profiles and reduced side effects would be a useful addition to the art. It would also be desirable to have vasopeptidase inhibitors in formulations which exhibit a longer half-life over conventional formulations which may lead to a more balanced NEP inhibition leading to higher trough/peak ratio for blood pressure reduction. Furthermore, vasopeptidase inhibitors that have a desired ratio of inhibition constants for ACE and NEP coupled with a longer half-life may prevent a surge in NEP inhibition initially and provide balanced ACE and NEP inhibition throughout the day.

U.S. Pat. No. 5,508,272 to Rob1 discloses compounds containing a fused bicyclic ring which are useful as dual action inhibitors, namely as angiotensin converting enzyme (ACE) inhibitors and as neutral endopeptidase (NEP) inhibitors, which compounds include omapatrilat.

U.S. Pat. No. 5,552,397 to Karanewsky et al. discloses substituted azepinone dual action inhibitors of ACE and NEP, which covers gemopatrilat.

### SUMMARY OF THE INVENTION

In accordance with the present invention, formulations for the controlled- or modified-release of vasopeptidase inhibitors at desired sites of absorption (namely, the stomach, small intestine and colon) have been found to provide more balanced release of drug within the first 4 to 6 hours of drug release as compared to immediate-release formulations. The modified-release formulations of the invention also provide for an improved balance of NEP inhibition profile over a predetermined dosing interval (as compared to immediate- or rapid-release formulations) whereby undesirable surge or high peak levels of NEP inhibitory activity over the first few hours of release is significantly diminished or blunted and a more gradual rise in plasma drug concentration is produced over the first few hours of drug release employing the modified-release formulations of the invention as compared to that employing immediate-release formulations. This improved balance in NEP inhibition profile is obtained without significantly adversely affecting the ACE inhibition profile obtained employing immediate-release formulations. Thus, the modified-release formulations of the invention may provide for a more modulated blood pressure lowering as compared to that obtained with immediate-release formulations.

The modified-release formulations of the invention exhibit ACE and NEP inhibition profiles which are more closely aligned in their altered pharmacokinetic profile and duration, and which have improved pharmacodynamic effects. In addition, ACE inhibition and NEP inhibition produced by the modified-release formulations of the invention are similar to that obtained with immediate-release formulations indicating preservation of pharmacodynamic effects despite modifying release of the drug. Furthermore, the modified-release formulations of the invention as compared to immediate-release formulations provide for improved efficacy and modulation in blood pressure lowering employing once a day dosing, reduction in peak levels and reduction in overall exposure of the vasopeptidase inhibitor to systemic circulation, improvement in trough/peak ratios (reduced peak while maintaining trough) for blood pressure lowering, reduction in dosing frequency and/or titrations (improved dosing regimen),and better patient compliance. In addition, the formulations of the invention provide for improvement in patient tolerability, that is use of the modified-release formulations of the invention may allow for reduced number of titrations required in arriving at a safe and effective dosage as compared to number of titrations required when employing immediate-release formulations. In fact, a patient should be able to tolerate a modified-release formulation of the invention containing 20 mg of omapatrilat in a manner equivalent to that of an immediate-release formulation containing 10 mg of omapatrilat.

### DESCRIPTION OF THE INVENTION

The modified-release formulation of the invention, which is preferably in a solid dosage form such as tablets or beads, includes a pharmaceutical capable of providing both NEP inhibitory activity and ACE inhibitory activity, and a modified-release drug delivery system therefor, whereby the drug delivery system allows for a more balanced release of the pharmaceutical, over the first 4 to 6 hours of drug release, at the desired site of absorption, in a manner to provide balanced therapeutically effective NEP inhibitory activity and balanced therapeutically effective ACE inhibitory activity over a desired period, as compared to prior art immediate-release formulations.

In preferred embodiments of the formulation of the invention, the once-a-day profile of NEP inhibitory activity is improved or balanced (that is provides for substantially reduced surges or peaks in drug release and substantially reduced or blunted surges or peaks in NEP inhibitory activity over the first few hours of drug release) to more closely resemble a formulation suitable for a once-a-day profile.

The modified-release formulation of the invention will beneficially modify the pharmacokinetic profile and the pharmacodynamic response of vasopeptidase inhibitors. It has been found that the modified-release formulation of the invention modulates pharmacodynamics of one pathway of action (that is, the NEP inhibitory pathway), while having minimal impact on the second pathway of action (that is, the ACE inhibitory pathway) to produce unexpected benefits over comparable immediate-release formulations, namely improved balance of blood pressure lowering over a desired period, reduction in the peak level (a C^{max} of from about 20 to about 80% of a comparable immediate-release formulation so that the C^{max} of modified-release formulation containing 20 mg of drug will be substantially comparable to or less than an immediate-release formulation containing 10 mg of drug), without substantial loss in NEP/ACE inhibitory activity, reduction in overall exposure of the vasopeptidase inhibitor to systemic circulation, a balance in the NEP inhibition profile, improvement in trough/peak ratio (reduced peak while maintaining trough) for blood pressure lowering, reduction in dosing frequency and number of titrations (improved dosing regimen), and improvement in patient compliance and tolerability.

In addition, in accordance with the present invention, a method is provided for enhancing or improving the balance of vasopeptidase inhibitor release, and improving the balance of NEP inhibitory activity profile of a vasopeptidase inhibitor for use in animal or human patients, preferably to be more closely aligned with the profile of its ACE inhibitory activity, as obtained in comparable immediate-release formulations, which method includes the step of formulating the vasopeptidase inhibitor in a modified-release formulation as described herein. The so-formed modified-release composition will be capable of releasing therapeutically effective amounts of vasopeptidase inhibitor, in a balanced manner so as to avoid or minimize initial surges or peaks in drug release and in NEP inhibitory activity and ACE inhibitory activity, in a patient over a desired period or dosing interval, preferably over a 24 hour period, while providing for both therapeutically effective NEP inhibitory activity and therapeutically effective ACE inhibitory activity essentially over the desired dosing interval.

In a preferred embodiment, the method will provide once-a-day dosing.

Still further in accordance with the present invention, a method is provided for reducing blood pressure or for treating congestive heart failure or other diseases as described hereinafter, in a mammalian patient, such as a human, dog or cat, wherein a modified-release formulation (as described herein) containing a compound capable of providing therapeutically effective amounts of both ACE inhibitory activity and NEP inhibitory activity, in a balanced manner, the compound preferably being a vasopeptidase inhibitor, is administered to a patient in need of treatment to effect reduction in blood pressure and/or treat congestive heart failure or other cardiovascular and related diseases over a desired dosing interval, preferably from about 4 to about 24 hours or more, more preferably from about 12 to about 24 hours.

In carrying out the method of the invention, the modified-release formulation will preferably be administered once a day to provide the required daily dosage of vasopeptidase inhibitor for the desired duration. The vasopeptidase inhibitor will be delivered, in a substantially balanced manner, at the desired site of absorption, for example, stomach, the small intestine, and large intestine, over the desired dosing interval providing both substantially balanced levels of therapeutically effective NEP inhibitory activity and ACE inhibitory activity substantially over the desired period as well as reduced peak levels, and improved trough/peak ratio as compared to immediate-release compositions containing such vasopeptidase inhibitor.

The modified-release formulation of the invention which contains a vasopeptidase inhibitor, is effective in preventing, reducing and/or treating elevated blood pressure levels, and/or heart failure, and other cardiovascular and related diseases including abnormalities in intraocular pressure, complications of diabetes, atherosclerosis, cardiovascular events and diseases including coronary events and cerebrovascular events, and coronary artery disease and/or cerebrovascular disease and/or angina and/or renal diseases such as renal failure, progressive or chronic renal failure, diabetic nephropathy, nephropathy, proteinuria and the like. However, it is to be understood that the formulations of the invention may be employed for preventing and/or treating any disease or condition which may be prevented and/or treated with ACE inhibitors and/or NEP inhibitors and/or vasopeptidase inhibitors.

The pharmaceutical employed in the modified-release formulation and method of the invention is preferably a member of the class of vasopeptidase inhibitors.

Preferred vasopeptidase inhibitors for use in the modified-release formulation of the invention are omapatrilat, gemopatrilat, CGS 30440 and MD 100,240.

Other examples of vasopeptidase inhibitors suitable for use herein include, but are not limited to, those disclosed in U.S. Pat. Nos. 5,362,727, 5,366,973, 5,225,401, 4,722,810, 5,223,516, 4,749,688, 5,552,397, 5,504,080, 5,612,359, and 5,525,723, European Patent Applications 0599,444, 0481,522, 0599,444, 0595,610, European Patent Applications 0534363A2, 534,396 and 534,492, and European Patent Application 0629627A2, and includes any compound which exhibits both NEP inhibitory activity and ACE inhibitory activity.

The drug delivery systems suitable for use in the modified-release formulation of the invention include, but are not limited to, controlled matrix systems including diffusion-controlled systems, dissolution controlled matrices, erodible/degradable systems, osmotic systems, and barrier membrane systems, as well as transdermal systems, buccal systems, inhalation systems, suppositories (rectal or vaginal) as well as parenteral and liquid delivery systems.

It will be appreciated that any conventional drug delivery system may be employed in the modified-release delivery system of the invention which will deliver vasopeptidase inhibitor to the desired sites of absorption in the body, for example, the stomach and/or small intestine, as well as the colon, and which will release therapeutically effective amounts of vasopeptidase inhibitor including therapeutically effective levels of both NEP inhibition and ACE inhibition, in a substantially balanced manner, preferably of similar duration, over the desired dosing interval, preferably over a period of at least 3 hours up to 24 hours.

The modified-release formulation of the invention will preferably be formulated in a solid dosage form such as tablets, beads, beadlets, pellets, granules, powders, capsules and the like, as well as transdermal systems, intranasal systems, suppositories (rectal and vaginal), as well as liquid dosage forms including elixirs, and parenteral forms.

The solid dosage formulation of the invention may include from about 0.1 to about 500 mg of vasopeptidase inhibitor and will preferably be administered in a daily dose once-a-day or can be administered in divided doses 2 to 4 times per day.

In one embodiment of the present invention, a modified-release coated vasopeptidase formulation is provided which allows for release of drug at the desired sites of absorption. The modified-release formulation of the invention is preferably in the form of beads or pellets, each bead of which includes a core which includes the vasopeptidase inhibitor, and a modified release coating surrounding the core.

It will be appreciated by those skilled in the art that corresponding and proportional levels of modified-release coating will be employed with different size beads.

The modified-release coating allow for release of vasopeptidase inhibitor at the desired sites of absorption, for example in the stomach and small intestine or colon to be absorbed therein. In fact, it is believed that the vasopeptidase inhibitor is primarily absorbed in the stomach and throughout the small intestine, namely, in the duodenum, jejunum, and ileum. It is also possible that the vasopeptidase inhibitor depending on its type, and/or the nature of the delivery system may be absorbed in the colon.

The resulting formulation of the invention provides for reduced peak levels and increased trough/peak ratios, decreased blood levels and balanced therapeutic action as compared to comparable immediate- or rapid-release compositions which have the stomach and duodenum as the major absorption site.

The modified-release vasopeptidase inhibitor formulation of the invention may comprise a plurality of modified-release coated beads (in the same capsule or in two or more capsules), the beads containing the same or varying and different levels or amounts of modified-release coating (that is a mixture of beads of the same or different bead size and containing the same or different modified release coating levels or amounts), the mixture of beads being contained in the same capsule or divided up into two more capsules, to enable the beads to be absorbed at the desired sites of absorption as described above.

The modified-release coating will include one or more modified release polymers which controls rate of drug release, preferably, one or more methacrylic acid copolymers, more preferably a copolymer of methacrylic acid and methacrylic acid methyl ester or a methylmethacrylate/methacrylic acid copolymer. The enteric polymer may be partially neutralized. In addition, the formulation will preferably include one or more hydrophobic plasticizers for the modified-release coating material, such as triethyl citrate and/or diethyl phthalate, and optionally one or more other excipients used for processing such as one or more wetting agents, optionally one or more antifoam agents and an optional overcoating of an anti-adherent.

The ability of the modified-release coat to allow for and enable release of vasopeptidase inhibitor at the desired sites of absorption, for example, for absorption in the stomach and small intestine, is important to achieving maximum improvement in bioavailability and blood levels including improvement in balance of NEP inhibitory activity, reduction in peak blood levels, and improvement in trough/peak ratios.

In a preferred embodiment of the invention, the modified-release coated beads will be formed of a mixture of beads which include varying and different amounts of modified-release coating so as to enable the beads to release vasopeptidase inhibitor substantially throughout the desired sites of absorption.

It will also be understood that the present invention includes a mixture of two or more beads of different dimensions each of which may have the same modified-release coating level. The beads of varying size, but having the same (or different) modified-release coating levels, will achieve prolonged release of vasopeptidase inhibitor.

Preferred formulations of the present invention may take the form of several embodiments. Thus, in one embodiment of the invention, a vasopeptidase inhibitor formulation is provided in the form of a plurality of beads which includes a vasopeptidase inhibitor-containing core and a modified-release coating as described above surrounding the core. In an optional embodiment, the core may include a protective seal or subcoating under the modified-release coat and/or an outer coating of an anti-adherent material. The beads may be loaded into capsules for dosing.

The above modified-release coated formulations of the invention may be employed in admixture or combination with known vasopeptidase inhibitor formulations including immediate- or rapid-release formulations.

In another embodiment of the present invention, there is provided a modified-release coated vasopeptidase inhibitor tablet composition which is formed of granules, particles or pellets of vasopeptidase inhibitor coated with a modified-release coating, which coated masses are compressed into tablets.

The modified-release matrix for the tablet formulations of the invention (which will be described in more detail hereinafter) will be formed of one or more hydrophilic polymers which modify or control rate of drug release, preferably hydroxypropyl methyl cellulose (for example, Methocel K4MPRMCR and Methocel K100LVPCRLH), and optionally one or more excipients including one or more soluble fillers and/or one or more insoluble fillers and/or tabletting aids, optionally one or more lubricants used for processing and/or optionally one or more anti-adherents used for processing.

In still another embodiment of the invention, there is provided a capsule composition wherein the capsule contains one or more modified-release coated matrix tablets or modified-release coated granules and optionally one or more immediate release tablets, granules, and/or beads, and combination of beads and bilayered tablets. The modified-release coating in this embodiment of the invention is preferably one or more hydrogels which are preferably formed of one or more hydrophilic polymers.

The modified-release formulation of the invention may also take the form of a modified-release matrix delivery system which includes a heterogeneous two phase system which includes (1) an inner solid particulate phase in the form of individual granules or particles containing (a) vasopeptidase inhibitor, and optionally (b) an extended release material formed of one or more hydrophilic polymers, and/or one or more hydrophobic polymers, and/or one or more other type hydrophobic materials (such as one or more waxes, fatty alcohols and/or fatty acid esters), and (2) an outer solid continuous phase in which granules or particles of inner solid particulate phase are dispersed and embedded, the outer solid continuous phase which primarily is formed of an extended release material formed of one or more hydrophilic polymers, and/or one or more hydrophobic polymers, and/or one or more other type hydrophobic materials (such as one or more waxes, fatty alcohols and/or fatty acid esters).

The modified-release matrix formulation of the invention is particularly adapted for delivery of vasopeptidase inhibitor in controlled and extended manner without significant initial burst of drug, and wherein release of drug (liberated from the individual dispersed particles forming the inner solid particulate phase) is effectively controlled. Drug upon being released from the particles of the inner phase, in effect, migrates through the outer solid continuous phase and then is released from the formulation throughout the gastrointestinal tract to be available for absorption.

The inner solid particulate phase will be formed of individual discrete particles or granules each of which contains vasopeptidase inhibitor and optionally one or more polymeric materials and/or other hydrophobic-type materials. In effect, the components of the inner solid particulate phase are in particulate association without having a barrier layer around the individual particles or granules.

The outer solid continuous phase is preferably a continuous phase or matrix having the particles or granules including drug (forming the inner solid phase) dispersed throughout and embedded in the continuous outer solid phase.

### DETAILED DESCRIPTION OF THE INVENTION DEFINITION OF TERMS

The terms “cardiovascular event(s)” and “cardiovascular disease”, and “cardiovascular and related diseases” as employed herein refer to coronary and/or cerebrovascular event(s) and disease including primary myocardial infarction, secondary myocardial infarction, myocardial ischemia, angina pectoris (including unstable angina), congestive heart failure, sudden cardiac death, cerebral infarction, cerebral thrombosis, cerebral ischemia, transient ischemic attack and the like, as well as any disease or condition that can be treated by ACE inhibitors, NEP inhibitors and/or vasopeptidase inhibitors.

The term “coronary artery disease” (CAD) as employed herein refers to diseases including atherosclerosis of the coronary arteries, previous myocardial infarction, ischemia, angina pectoris and/or heart failure.

The term “cerebrovascular disease” as employed herein refers to diseases including atherosclerosis of the intracranial and/or extracranial arteries, cerebral infarction, cerebral thrombosis, cerebral ischemia, stroke, and/or transient ischemic attacks.

The term “diabetic complications” as employed herein refers to diabetic neuropathy, diabetic retinopathy, diabetic nephropathy, atherosclerosis and other maladies caused as a result of diabetes.

Unless otherwise indicated, the terms “controlled-release”, “sustained-release” and “modified-release, as employed herein are used interchangeably.

Unless otherwise indicated, the terms “vasopeptidase inhibitor” and “NEP/ACE inhibitor” and ACE/NEP inhibitor, “dual action inhibitor”, “dual inhibitor”, “neutral metalloprotease inhibitor”, “and dual action metalloprotease inhibitor”, as employed herein are used interchangeably.

The term “absorbed substantially throughout the stomach, small intestine and large intestine (or colon)” refers to the ability of the vasopeptidase inhibitor to be absorbed not only in the stomach and duodenum but down to the jejunum and the ileum and the colon, where substantial amounts of vasopeptidase inhibitor are absorbed as well.

The term “mixture of beads containing different levels of modified-release coating” refers to the fact that the vasopeptidase inhibitor bead formulation of the invention includes a mixture of beads, which may be the same or different sizes, preferably the same size, prescribed portions of which are coated with prescribed levels of modified-release coating (which may include different levels of modified-release coating) to enable beads to be released and absorbed in the stomach and throughout the small intestine, that is in the duodenum, jejunum and ileum as well as in the colon, not primarily in the stomach and duodenum as in the case of immediate release vasopeptidase inhibitor formulations. The various portions of beads containing different modified-release coating levels may also contain the same or different amounts of vasopeptidase inhibitor.

The term “enhanced absorption and/or bioavailability properties” of vasopeptidase inhibitor refers to the ability of the vasopeptidase inhibitor to be absorbed into the blood stream of a human patient by delivering it to the desired site, for example stomach and the small intestine and colon to provide prolonged NEP inhibitory activity in spite of decreased AUC due to shift in Tmax.

The term “balanced . . . release” or “balanced . . . activity” refers to release of drug to provide NEP inhibitory activity and/or ACE inhibitory activity with reduced incidence of surges or peak levels during the first few hours of release at the desired sites of absorption as compared to that obtained with immediate release formulations.

The terms “particles”, “granules”, “pellets”, “beadlets”, and “beads” of vasopeptidase inhibitor are used interchangeably, and will preferably be from about 0.05 to about 10 mm in diameter.

The term “vasopeptidase inhibitor” as employed herein encompasses omapatrilat, gemopatrilat, CGS 30440, MD100, 240 and other vasopeptidase inhibitors as described herein and/or known in the art.

The term “comparable immediate-release or rapid-release formulations” refers to prior art immediate or rapid release formulations which contain amounts of vasopeptidase inhibitor essentially equivalent to that contained in the formulation of the invention.

The term “extended release material” as optionally present in the inner solid particulate phase and present in the outer solid continuous phase refers to one or more hydrophilic polymers and/or one or more hydrophobic polymers and/or one or more other type hydrophobic materials, such as, for example, one or more waxes, fatty alcohols and/or fatty acid esters. The “extended release material” present in the inner solid particulate phase may be the same as or different from the “extended release material” present in the outer solid continuous phase. However, it is preferred that the “extended release material” present in the inner solid particulate phase be different from the “extended release material” present in the outer solid continuous phase.

### BRIEF DESCRIPTION OF FIGURES

FIG. 1 is a chart showing plasma omapatrilat concentration (ng/mL) versus time of release comparing Control A immediate-release omapatrilat tablets and Examples 1 and 2 modified-release omapatrilat tablets.

FIG. 2 is a chart showing ACE-activity versus time of release comparing Control A immediate-release omapatrilat tablets and Examples 1 and 2 modified-release omapatrilat tablets.

FIG. 3 is a chart showing urinary ANP excretion (a marker of tissue NEP inhibition) versus time of release comparing Control A immediate-release omapatrilat tablets and Examples 1 and 2 modified-release omapatrilat tablets.

FIG. 4 is a chart showing plasma gemopatrilat concentration (ng/mL) versus time of release comparing Control B immediate-release gemopatrilat tablets and Example 3 gemopatrilat beadlets, and Examples 4 and 5 modified-release gemopatrilat tablets.

FIGS. 5A and 5B are charts showing ACE-activity versus time of release comparing Control B immediate-release gemopatrilat tablets and (1) Examples 4 and 5 modified-release gemopatrilat tablets (FIG. 5A) and (2) Example 3 modified-release gemopatrilat beadlets (FIG. 5B).

FIGS. 6A and 6B are charts showing urinary ANP excretion (a marker of tissue NEP inhibition) versus time of release comparing Control B immediate-release gemopatrilat tablets and (1) Examples 4 and 5 modified-release gemopatrilat tablets (FIG. 6A) and (2) Example 3 modified-release gemopatrilat beadlets (FIG. 6B).

FIGS. 7A and 7B are charts showing the hourly changes in ambulatory blood pressure (diastolic (ADBP) FIG. 7A and systolic (ASBP) FIG. 7B) vs. baseline after four weeks treatment with omapatrilat modified release tablets of the invention and immediate release control tablets.

Preparation of Granule or Bead Cores

In preparing the above described oral formulations, granules, particles or beads of vasopeptidase inhibitor can be incorporated into the formulation. In many cases, discrete granules of vasopeptidase inhibitor are needed so that these can be coated with a coating layer. The process of making vasopeptidase inhibitor granules may be common to most of the formulations. The granules or beads may be prepared in different ways and used in any of the above formulations depending on the specific needs and limitations of the formulation. Some of the processes which can be used for preparing granule formulations are described below.

(a) The granules or beads of vasopeptidase inhibitor for use in preparing tablets or capsules can be formed by dry compacting the vasopeptidase inhibitor as is or after blending with a soluble filler, such as anhydrous lactose, lactose monohydrate, cornstarch, modified cornstarch and/or mannitol, in an amount within the range from about 10 to about 70% by weight, preferably from about 20 to about 60% by weight; insoluble filler and/or tabletting aid such as microcrystalline cellulose, wood cellulose, dicalcium phosphate, calcium carbonate, calcium sulfate, dextrin/dextrates, maltidextrin, sorbitol, compressible sugars, xylitol, and/or mixtures of 2 or more thereof, in an amount within the range from about 5 to about 65% by weight, a lubricant, such as zinc stearate, magnesium stearate, stearic acid, sodium stearyl fumarate, calcium stearate, talc, carnauba wax, or hydrogenated vegetable oils and/or fats, in an amount within the range from about 0.01 to about 5%, and preferably from about 0.1 to about 4%; all of the above % being based on the weight of the uncoated granule or bead.

One or more binders will optionally be present in addition to or in lieu of the fillers in an amount within the range of from about 0 to about 35% and preferably from about 1 to about 30% by weight. Examples of such binders which are suitable for use herein include basified polyvinylpyrrolidone (molecular weight ranging from about 5000 to about 80,000 and preferably about 40,000), lactose, HPMC, starches such as corn starch, modified corn starch, sugars, gum acacia and the like as well as a wax binder in finely powdered form (less than 500 microns) such as carnauba wax, paraffin, spermaceti, polyethylenes or microcrystalline wax.

The compacts, granules or beads can be prepared by roller compaction or slugging. By this process, granules of almost 100% drug load can be prepared. Lower drug load granules may be prepared by employing additional fillers and excipients in the blend used for compaction. The compacts can be broken into granules with suitable equipment. The resulting granules can be sized and the desired size fraction can be separated and collected with the rest being recycled.

(b) Spherical and very high drug load core granules or core beads of vasopeptidase inhibitor can be prepared by simple wet granulation or micro-granulation of vasopeptidase inhibitor in a high shear granulator using water. Sufficient water or granulating solution containing a binder is added to enable preparation of small spherical granules (that is, average particles size of less than about 1 mm). The resulting granules can be sized and screened to collect desired size fraction, and dried to desired moisture level. The under and over sized fractions can be recycled. If less than 100% drug load is desired, fillers and excipients can be included in the powder to be micro granulated.

(c) In another embodiment of the above method, vasopeptidase inhibitor granules or beads can be prepared using Moisture Activated Dry Granulation (MADG) process. In this case, a portion (30-60%) of the vasopeptidase inhibitor core can be granulated as above using all of the moisture needed for the whole blend to form agglomerates and then the remaining vasopeptidase inhibitor added, and the mixture blended to prepare the granules. The final blend can be sized, screened, and desired size fraction removed with over and under size to be recycled if necessary. In this process, since normally, drying is not involved, minimum quantities of moisture will be used as compared to process (b). However, for stability reasons, the granules so prepared can still be dried.

(d) In yet another embodiment of the above method, conventional wet granules of the vasopeptidase inhibitor as in process (b) can be prepared by first making a wet mass using conventional wet massing equipment. The wet mass can be sized wet and dried or dried as is and then broken into granules, which are screened and the desired size fraction is collected.

(e) In still another embodiment of the above method, the wet mass prepared in method (d) can be extruded. The extrudate can be dried, broken into granules, and sieved to collect desired size fraction. This process can produce high (>90%) drug load, dense and hard particles.

(f) In yet another embodiment of the above method, more uniform and spherical particles can be prepared by employing conventional spheronization processes. This will require use of higher level of excipients to allow proper extrusion of the wet mass and trouble free spheronization of the extrudate. Depending on the selection of the excipients and modification of the spheronization process, it may require 5 to 99% excipients. For this purpose, vasopeptidase inhibitor (representing from about 1 to about 95% by weight of the final core bead (or core), preferably from about 15 to about 30% by weight of the final core bead) is blended with microcrystalline cellulose and/or other insoluble spheronizing aid and/or insoluble fillers such as set out above in process (a). The blend is then wet massed and extruded. The extrudate is then spheronized to prepare the beads. These beads are dried in a hot air tray oven or fluid bed dryer. The beads can be further sized to remove under and over sized particles. While microcrystalline cellulose is preferred for bead formation, other excipients, for example, starch, lactose, starch 1500, silicified microcrystalline cellulose and the like, or any combination of these may be used as well.

(g) In yet another embodiment of this method, a much higher drug concentration bead can be prepared by saving a portion of the drug blend before wet massing and using this dry powder to dust while spheronizing.

Preparation of Modified Release Coated Cores Including Beads and Tablets

The vasopeptidase inhibitor formulation of the invention in the form of a plurality of modified-release coated, vasopeptidase inhibitor particles, tablets, beadlets, beads, or cylindrical particles, may be prepared by first forming vasopeptidase inhibitor granules, particles, beads, granules or cylindrical particles (hereinafter “granules”) employing any of the granulation processes described above, preferably process (f) for beads or process (a) for granules. These granules, which will have an average particle size within the range from about 20 μm to about 2 mm, can be coated with an optional protective seal or subcoat, for example, employing a hydrophilic coating polymer such as a 2-10% aqueous solution of polyvinyl pyrrolidone (PVP), a 2-20% aqueous solution of hydroxypropylmethyl cellulose (HPMC), or Opadry Clear (HPMC), or a 10-30% suspension of neutralized Eudragit L-30-D55 (acrylic acid copolymers-Rohm America Incorporated) (about 30% solids), alginates, cellulose acetate, cellulose acetate phthalate, ethyl cellulose, hydroxypropylmethyl cellulose (all grades of Methocel A, F, E and K), derivatives of HPC and HPMC (such as hydroxypropyl cellulose, HPMC phthalate), polymethacrylates and xanthan gum, in an amount from about 60 to about 100% by weight of the subcoat, preferably from about 70 to about 100% by weight) and containing 10 to 40% (preferably 10 to 17% based on weight of subcoat) plasticizer such as diethyl phthalate (W/W) or Citroflex®, polyethylene glycol, dibutyl sebacate, glycerin, glyceryl monostearate, mineral oil and lanolin alcohols, petrolatum and lanolin alcohol, propylene glycol, triacetin and triethyl citrate. A 0.5 to 10% (based on weight of final modified-release coated granule) protective coating may be applied in a fluid bed particle coating system or a coating pan.

The vasopeptidase inhibitor beads with or without the protective coating are coated with a modified-release coating. The modified-release coated beads can be further coated with an anti-adherent coating. These beads can be dosed as granules or beads or in the form of capsules after encapsulation. Upon ingestion, the beads will be delivered to the desired sites of absorption. For example, as the beads reach the stomach and the duodenum, jejunum and ileum, the modified-release coat will dissolve followed by dissolution of the vasopeptidase inhibitor particles and absorption thereby at the desired sites of absorption.

The vasopeptidase inhibitor formulation of the invention will contain vasopeptidase inhibitor in an amount within the range from about 0.5 to about 95% by weight of the formulation, preferably from about 1 to about 40% by weight. Thus, depending upon the particular vasopeptidase inhibitor, it may be employed in amounts within the range from about 0.5 mg to 2000 mg per day in single or divided doses, and preferably from about 1 to about 400 mg per day. Most preferably for omapatrilat and gemopatrilat, a daily dosage of 5 to 160 mg may be employed, preferably once daily.

The modified-release coating will be present in varying and different levels as described herein to impart the desired properties to such formulation as described hereinbefore. The modified-release coating will be one or more modified release hydrophilic polymers, preferably methacrylic acid copolymer, a copolymer of methacrylic acid and methacrylic acid ester or a copolymer of methylmethacrylate/methacrylic acid (and preferably a mixture of Eudragit RL30D-and/or Eudragit RS-30D, ammonio methacrylate copolymers, Rohm America Incorporated). Other modified-release polymers suitable for use herein include but are not limited to carboxymethylcellulose sodium, cellulose acetate, cellulose acetetate phthalate, ethylcellulose, hydroethyl cellulose, hydroxypropyl cellulose, hydroxypropylmethyl cellulose, hydroxyypropyl-methylcellulose phthalate, methylcelulose, polymethacrylates, shellac, waxes (i.e. carnauba wax, microcrystalline wax, white wax, and/or yellow wax), xanthan gum, and zein. The modified-release polymer will be present in an amount from about 10 to about 95% by weight of the enteric coating. The enteric polymer may be partially neutralized.

The modified-release coating will include a plasticizer preferably a hydrophobic plasticizer in an amount from about 0 to 50%, preferably from about 0.5 to about 35% by weight of the modified-release coating. Examples of plasticizers include diethyl phthalate, tributyl citrate, triacetin, glycerin, glyceryl monostearate, mineral oil or petrolatum and lanolin alcohols, PEG, propylene glycol, dibutyl phthalate, dibutyl sebacate, or Myvacet 940 (acetylated monoglycerides) and other commonly used plasticizers, preferably triethyl citrate or diethyl phthalate as may be suitable for the enteric polymer employed herein. It will be appreciated that the modified-release polymer with suitable plasticizer can be used in aqueous or non-aqueous system to form a modified-release coating on the vasopeptidase inhibitor bead or granule.

At least 35 grams of the coating solution per m² surface area would be sufficient to provide a coat with modified-release qualities when applied from an aqueous system.

In a preferred embodiment of the invention, to form the modified-release coating, a 10-30% suspension of Eudragit® RL30D and RS30D ammonio methacrylate copolymers, preferably 15 to 20%, containing 1 to 5% triethyl citrate or diethyl phthalate plasticizer, preferably 1.5-3.0% is prepared in purified water. The coating suspension may also optionally include an anti-adherent agent in an amount from about 0 to about 60% by weight of the enteric or film coat, preferably from about 10 to about 50% by weight, such as silicon dioxide, magnesium silicate, talc, as well as any of the following: fumaric acid, glyceryl monostearate, glyceryl palmitostearate, isopropyl myristate, magnesium stearate, medium chain triglycerides, mineral oil, poloxamer, polyethylene glycol, polyoxyethylene stearates, sodium lauryl sulfate, sodium stearyl fumarate, stearic acid, titanium dioxide, vegetable oil, and zinc stearate. In addition, the suspension may optionally include one or more wetting agents in an amount from about 0 to about 10% by weight of the enteric or film coat, such as polysorbate 80, docusate sodium, poloxamer, polyoxyethylenes (i.e. polyoxyethylene alkyl ethers, polyoxyethylene castor oil derivatives, polyoxyethylene sorbitan fatty acid esters, and polyoxyethylene stearates) sodium lauryl sulfate and sorbitan esters (sorbitan fatty acid esters).

The uncoated or protective-coated cores or beads are modified-release coated with this suspension in a fluid bed coater fitted with a Wurster column or top coating capability or a pan-coater.

The above modified-release coated granules may be further coated with an overcoat of an anti-adherent material such as silicon dioxide, talc, magnesium stearate, calcium stearate, silica gel, magnesium silicate, titanium dioxide as well as fumaric acid, glyceryl monostearate, glyceryl palmitostearate, isopropyl myristate, medium chain triglycerides, mineral oil, poloxamer, polyethyl glycol, polyoxyethylene stearates, sodium lauryl sulfate, sodium stearyl fumarate, stearic acid, titanium dioxide, vegetable oil, and zinc stearate. The anti-adherent material, preferably talc or SiO₂, is used at 0.1-10% level and preferably 0.2 to 8.0% based on the weight of the final modified-release coated granules. Coated particles and talc may be loaded into a tumbling type blender and blended for 5-30 minutes.

The above coated particles can be encapsulated into hard gelatin capsules or compressed into tablets for the desired potency.

A preferred modified- or controlled-release vasopeptidase granule or bead (contained in a capsule) formulation of the invention which may be prepared by the above method is set out below.

| | Possible Range | Preferred Range |
|---|---|---|
| Materials | % by Weight | % by Weight |
| Vasopeptidase | 0.5 to 95% | 1 to 40% |
| inhibitor | | |
| Soluble filler such | 10 to 70% | 20 to 60% |
| as anhydrous | | |
| lactose | | |
| Insoluble filler | 5 to 65% | 10 to 35% |
| such as | | |
| microcrystalline | | |
| cellulose | | |
| Modified-Release | 5 to 65% | 10 to 45% |
| Hydrophilic Polymer | | |
| such as HPMC | | |
| Plasticizer such as | 0 to 40% | 4 to 30% |
| triethyl citrate | | |
| Lubricant such as | 0.1 to 5% | 1 to 4% |
| stearic acid | | |
| Anti-adherent such | 0.1 to 5% | 0.2 to 4% |
| as SiO₂ or Talc | | |

* % by weight based on the total weight of the final tablet or granule.

A preferred modified-release coated vasopeptidase inhibitor formulation of the invention in the form of a bead which include optional subcoat and overcoat is set out below.

| | Possible Range | | Preferred Range | |
|---|---|---|---|---|
| Material | % by weight | | % by weight | |
| Core Beads % based on | 40 to | 95 | 75 to | 90 |
| Final Coated Bead | | | | |
| Vasopeptidase | 1 to | 90 | 50 to | 80 |
| inhibitor (% based on | | | | |
| core bead) | | | | |
| Spheronizing Aid or | 10 to | 99 | 20 to | 50 |
| Filler such as | | | | |
| Microcrystalline | | | | |
| cellulose (% based on | | | | |
| core bead) | | | | |
| Optional Subcoat | (0 to | 35) | (1.8 to | 17.5) |
| (% based on Final | | | | |
| Coated Bead) | | | | |
| Film polymer such as | 60 to | 100 | 70 to | 100 |
| Methocel E5 (HPMC) (% | | | | |
| based on weight of | | | | |
| subcoat) | | | | |
| Plasticizer such as | 0 to | 40 | 0 to | 17 |
| Polyethylene glycol (% | | | | |
| based on weight of | | | | |
| subcoat) | | | | |
| Modified-Release Film | (5 to | 30%) | (5 to | 20%) |
| Coat (% based on final | | | | |
| coated beads) | | | | |
| Modified-release | | | | |
| polymer such as | 20 to | 70% | 30 to | 60% |
| Polymethacrylic acid | | | | |
| esters such as | | | | |
| EUDRAGIT RL-30D and | | | | |
| RS-30D (% based on | | | | |
| weight of film coat) | | | | |
| Plasticizer such as | | | | |
| diethyl phthalate or | 2 to | 40% | 4 to | 30% |
| triethyl citrate (% | | | | |
| based on weight of | | | | |
| film coat) | | | | |
| Anti-adherent such as | | | | |
| Mg silicate or talc (% | 0% to | 50% | 20% to | 50% |
| based on weight of | | | | |
| film coat) | | | | |
| Wetting agent such as | | | | |
| polysorbate 80 (% | 0 to | 10% | 0.01 to | 1% |
| based on weight of | | | | |
| film coat) | | | | |
| Antifoaming agent such | 0 to | 10% | 0 to | 2% |
| as Antifoaxn Emulsion C | | | | |
| Overcoat (% based on | (0 to | 10%) | (0.1 to | 3.0%) |
| Final Coated Bead | | | | |
| Dusting Talc, Mg | | | | |
| silicate or SiO₂ (% | 0 to | 10% | 0.1 to | 3.0% |
| based on weight of | | | | |
| overcoat) | | | | |

The modified-release coat and the optional subcoat are based on the surface area of the core beads.

The modified-release coated formulation of the invention in the form of pellets or beadlets may also be prepared employing an extrusion-spheronization procedure such as described in U.S. Pat. No. 4,808,413 to Joshi et al. For example, the pharmaceutical is dissolved in a granulation liquid (water). The fillers, binders and disintegrants (for example, microcrystalline cellulose, lactose, sodium starch glycolate, polyvinylpyrollidone and sodium citrate) are thoroughly mixed, for example, using a conventional mixer such as a planetary mixer, to form a dry blend. The dry blend is then granulated using the above granulation solution and continued to the endpoint with water. The wet mass is extruded, for example, employing a Nica, Luwa or other type of extruders to form an extrudate which is then passed through spheronizing equipment, such as Nica, Caleva or other type, which converts the extrudate into beadlets of appropriate particle size range. The beadlets may then be dried by tray drying oven or fluid bed drying. Where the core is to be a tablet, the tablet may be formed using conventional techniques.

The dried beadlets or pellets, may then be coated with a subcoat, for example, with a solution of hydroxypropylmethyl cellulose (Pharmacoat 603) and polyethylene glycol 400. These sub-coated beadlets or pellets are then overcoated with the modified-release coating composition which is a dispersion of a copolymer of polymethacrylic acid esters and a plasticizer preferably diethyl phthalate.

The so-formed pellets or beadlets may be filled into hard gelatin capsules.

Omapatrilat and Gemopatrilat Modified-Release Capsule (Bead) Formulations

Omapatrilat and gemopatrilat capsule (bead) formulations of the invention which include granules or beads of drug, filler-binder, and modified-release polymer coating having the following composition is prepared as described below.

Formula for Capsule Formulation:

| Uncoated Omapatrilat or | 1-90% drug load | |
|---|---|---|
| Gemopatrilat Beads | | |
| (microcrystalline cellulose) | (10-99% by weight of | |
| | core beads) | |
| Optional Protective or Subcoating | | |
| Uncoated Omapatrilat or | 90 to 100 | mg |
| Gemopatrilat Beads | | |
| HPMC | 0 to 10 | mg |
| PEG | 0 to 2 | mg |
| Purified Water | q.s. | |
| Modified-Release Coating | | |
| Uncoated Omapatrilat or Gemopatrilat | 100 | mg |
| Beads or Protective Coated Omapatrilat | | |
| or Gemopatrilat Beads | | |
| Mixture of Ammonio Methacrylate | 3 to 10 | mg |
| Copolymer (Dry Weight Basis) | | |
| Triethyl Citrate (Plasticizer) | 0.5 to 10 | mg |
| Mg Silicate or Talc | 2 to 10 | mg |
| Purified Water | q.s. | |

Procedure:

A multi-step process is employed which starts with the preparation of omapatrilat or gemopatrilat granules. The granules can be prepared by any of the methods (a)-(f) described above, but methods (e) and (f) are preferred. These granules (having an average particle size ranging from about 710 μm to about 1700 μm) may optionally be coated with a protective coat. The protective coating polymer may be a 2-10% solution of basified PVP, or 2-20% solution of HPMC or Opadry® Clear with or without a suitable plasticizer such as polyethylene glycol. A 0.5-10% coating (based on weight of finished modified-release coated granule) can be applied in a fluid bed particle coating system. Among these, a preferred protective coating will be formed of 60 to 100% (based on weight of subcoating) HPMC. The beads or particles are coated to 0.5-10% coat level in fluid bed apparatus with or without a Wurster insert or in a pan coater.

The above granules are coated as described above. These are coated with modified-release coating polymers as follows. A 10-30% suspension of a mixture of Eudragit RL-30D, and RS-30D, preferably 15 to 20%, containing 1 to 6% diethyl phthalate or triethyl citrate preferably 1.5 to 4.5%, is prepared in purified water. The uncoated or protective coated beads are modified-release coated with this suspension in a fluid bed coater fitted with or without a Wurster column or in a pan coater. A 15% to 60% weight gain would be sufficient to provide coat with modified-release qualities.

The above modified-release coated granules are further coated with anti-adherent material such as talc, magnesium stearate, calcium stearate, silica gel or titanium dioxide. Preferably talc is used as anti-adherent at 0.1 to 4% level, preferably 0.5 to 2%. Modified-release coated granules and talc are loaded into a tumbling type blender and blended for 5 to 30 minutes, preferably 10 minutes.

The above coated granules are encapsulated into capsule hard shells suitable for the desired potency or compressed into a tablet matrix using a cushioning filler-binder system to provide a dosage form with 5 to 160 mg drug potency.

The resulting vasopeptidase formulation will provide for improved balance of ACE and NEP inhibitory activity in human patients over a similar formulation which does not contain the modified-release coating as defined above. When the beads with different coating levels are mixed, these provide a further prolonged release formulation.

Modified-Release Tablet (Matrix) Formulations

The modified-release tablet (matrix) system of the invention will include the inner solid particulate phase (containing the vasopeptidase inhibitor) in a weight ratio to the outer solid continuous phase within the range from about 0.05:1 to about 4:1, preferably from about 0.1:1 to about 1:1.

The inner solid particulate phase will contain vasopeptidase inhibitor in an amount within the range from about 0.5 to about 95% by weight, preferably from about 1 to about 40% by weight, and optional extended release material in the form of hydrophilic polymers and/or hydrophobic polymers and/or other hydrophobic material in an amount within the range from about 5 to about 65% by weight, preferably from about 10 to about 45% by weight, the above % being based on the weight of the inner solid particulate phase. Where mixtures are employed, the hydrophilic polymer will be employed in a weight ratio to hydrophobic polymer and/or other hydrophobic material within the range from about 0.05:1 to about 19:1, preferably from about 0.1:1 to about 10:1.

The particles or granules of the inner solid particulate phase will have a mean particle size within the range from about 1 μm to about 0.8 mm, and preferably from about 5 μm to about 0.5 mm.

The outer solid continuous phase will contain extended release material (preferably different from that employed in the inner solid particulate phase) in the form of one or more hydrophilic polymers and/or hydrophobic polymers and/or other hydrophobic material in an amount within the range from about 40 to about 100%, preferably from about 60 to about 100% (based on the weight of the outer solid continuous phase).

The outer solid continuous phase may contain mixtures of two or more extended release materials in the form of one or more hydrophilic polymer and/or hydrophobic polymer and/or other hydrophobic material in a weight ratio of hydrophilic polymer to hydrophobic polymer or other hydrophobic material within the range from about 200:1 to about 0.05:1, preferably from about 100:1 to about 0.1:1.

The pharmaceutical formulation of the invention will have a total optional polymer extended release material content (including hydrophilic polymers and/or hydrophobic polymers and/or other hydrophobic material present in the inner solid particulate phase and hydrophilic polymer and/or hydrophobic polymers and/or other hydrophobic material present in the outer solid continuous phase) within the range from about 10 to about 75% by weight, preferably from about 30 to about 65%, more preferably from about 35 to about 60% by weight based on the total weight of the pharmaceutical formulation.

Hydrophilic polymers which may be employed in the inner solid particulate phase and/or outer solid continuous phase include, but are not limited to hydroxypropylmethylcellulose, hydroxypropylcellulose, sodium carboxymethylcellulose, carboxymethylcellulose calcium, ammonium alginate, sodium alginate, potassium alginate, calcium alginate, propylene glycol alginate, alginic acid, polyvinyl alcohol, povidone, carbomer, potassium pectate, potassium pectinate, and the like.

Hydrophobic polymers which may be employed in the inner solid particulate phase and/or outer solid continuous phase include, but are not limited to ethyl cellulose, hydroxyethylcellulose, ammonio methacrylate copolymer (Eudragit RL™ or Eudragit RS™), methacrylic acid copolymers (Eudragit L™ or Eudragit S™), methacrylic acid-acrylic acid ethyl ester copolymer (Eudragit L 100-5™), methacrylic acid esters neutral copolymer (Eudragit NE 30D™), dimethylaminoethylmethacrylate-methacrylic acid esters copolymer (Eudragit E 100™), vinyl methyl ether/maleic anhydride copolymers, their salts and esters (Gantrez™).

Other hydrophobic materials which may be employed in the inner solid particulate phase and/or outer solid continuous phase include, but are not limited to waxes such as beeswax, carnauba wax, microcrystalline wax, and ozokerite; fatty alcohols such as cetostearyl alcohol, stearyl alcohol; cetyl alcohol and myristyl alcohol; and fatty acid esters such as glyceryl monostearate, glycerol monooleate, acetylated monoglycerides, tristearin, tripalmitin, cetyl esters wax, glyceryl palmitostearate, glyceryl behenate, and hydrogenated castor oil.

Where hydrophilic polymers and/or hydrophobic polymers are used in the inner solid particulate phase and/or the outer solid continuous phase, such polymers can be ionic or non-ionic, preferably ionic for the inner solid particulate phase and preferably non-ionic for the outer solid continuous phase.

Preferred ionic polymers for use in the inner solid particulate phase include sodium alginate, carbomer (Carbopol™), calcium carboxymethylcellulose, or sodium carboxymethylcellulose, xanthan gum, methacrylic acid-acrylic acid ethyl ester copolymer, dimethylaminoethylmethacrylatemethacrylic acid esters copolymer, cellulose acetate phthalate, hydroxypropylmethylcellulose phthalate, hydroxypropylmethylcellulose trimellitate, and hydroxypropylmethylcellulose maleate, with sodium carboxymethylcellulose being particularly preferred.

Preferred non-ionic polymers for use in the outer solid continuous phase are those which assure rapid hydration of the outer solid continuous phase to minimize a variable and significant burst of drug, yet effectively control the release of drug being liberated from the discrete particles or granules forming the inner solid particulate phase. The liberated drug will migrate through the non-ionic polymers forming the outer solid continuous phase before being released from the dosage form and being available for absorption. Preferred polymers for the outer solid phase with the appropriate hydration characteristics include hydroxypropylmethyl cellulose 2910 USP (hydroxypropylmethylcellulose with a methoxyl content of 19-24% and a hydroxypropyl content of 7-12%), viscosity grades ranging from about 4000 to about 100,000 cps and hydroxypropylmethylcellulose 2208 USP (hydroxypropyl-methylcellulose with a methoxyl content of 28-30% and a hydroxypropyl content of 7-12%), viscosity grades ranging from about 3 to about 150 cps. In particular preferred embodiments of the outer solid phase, the above preferred polymers are used in admixture in weight ratios of hydroxypropylmethylcellulose 2910 USP:hydroxypropylmethylcellulose 2208 USP within the range from about 25:1 to about 50:1, preferably from about 30:1 to about 40:1.

Preferred modified-release tablet (matrix) delivery systems in accordance with the present invention are as follows.

| | | % by Weight of Inner |
|---|---|---|
| A. | Inner Solid Particulate Phase | Solid Particulate Phase |
| | (1) Vasopeptidase inhibitor | 55 to 98 |
| | (preferably Omapatrilat or | |
| | Gemopatrilat) | |
| | (2) Optional Polymer or | 5 to 95 |
| | Hydrophobic Material | |
| | Preferred: ethylcellulose and/or | 5 to 45 |
| | sodium carboxymethylcellulose | |
| | and/or glyceryl monostearate | |

(Average Particle Size of granules
forming inner solid particulate
phase: 0.05 to 2.0 mm)

| | | | % by Weight of Inner |
|---|---|---|---|
| B. | Outer Solid Continuous Phase | | Solid Continuous Phase |
| | Material: | | 40 to 100 |
| | | Preferred | |
| | (1) | Hydroxypropylmethyl- | 60 to 100 |
| | | cellulose 2208 USP | |
| | | (100,000 cps) | |
| | (2) | Hydroxypropylmethyl | 1 to 30 |
| | | cellulose 2910 USP (5 cps) | |

| Weight Ratio of Inner Solid Phase: | | 0.5:1 to 1.5:1 |
|---|---|---|
| Outer Solid Phase | | |
| | | % by Weight of Outer |
| C. | Optional Ingredients | Solid Continuous Phase |
| | Lubricant or Antiadherent | 0.02 to 5 |
| | (e.g. Mg Stearate, stearic | |
| | acid or silicon dioxide) | |
| | Compression aid (e.g. | 0 to 30 |
| | Microcrystalline cellulose) | |

The modified-release matrix formulation of the invention may be prepared in accordance with the following method of the invention.

A mixture of vasopeptidase inhibitor, preferably omapatrilat, insoluble filler, soluble filler and hydrophobic polymer and a portion of the lubricant are mixed in a suitable blender.

The blend is dry granulated using a roller compactor. The resulting compacted ribbons are reduced to form granules using an appropriate screening device.

The resulting dry granules are blended with hydrophilic polymer and/or hydrophobic polymer and/or other hydrophobic material. The resulting mix usually with lubricant is pressed into tablets or filled into capsules.

The finished dosage form is either a compressed tablet or a hard gelatin capsule, preferably a tablet. The tablet may be optionally film coated. The total amount of drug per dosage unit would be such as to offer a dosage form of convenient size for patients.

Utility of Formulations

The formulations of the invention containing the vasopeptidase inhibitor are useful in the treatment of physiological conditions in which ACE inhibitors and/or NEP inhibitors have been shown to be useful. Such conditions include disease states characterized by abnormalities in blood pressure, intraocular pressure, and renin including cardiovascular diseases particularly hypertension and congestive heart failure, glaucoma, and renal diseases such as renal failure, diabetic nephropathy, and renal impairment following treatment with cyclosporine or other immunosuppressants. Other conditions in which angiotensin converting enzyme inhibitors have been reported to be useful include hepatic cirrhosis, inhibiting the progression of atherosclerosis, preventing or treating hypertensive or diabetic retinopathy, improving myocardial dysfunction during or following a myocardial infarction, and preventing restenosis after angioplasty. The formulations of the invention containing the vasopeptidase inhibitor are also useful in the treatment of physiological conditions in which neutral endopeptidase inhibitors have been shown to be useful. Such conditions also include cardiovascular diseases particularly hypertension, hyperaldosteronemia, renal diseases, glaucoma, as well as the relief of acute or chronic pain. Thus, the formulations of the invention containing the vasopeptidase inhibitor are useful in reducing blood pressure which may be due to their diuresis and natriuresis properties.

The dose administered must be adjusted according to age, weight and condition of the patient, as well as the route of administration, dosage form and regimen and the desired result.

Dosages and Administration

The formulations described can be administered for these effects in amounts similar to those employed previously for angiotensin converting enzyme inhibitors. For example, the composition of the invention can be administered to a mammalian host such as man at from about 0.01 mg. to about 20 mg. per kg. of body weight per day, preferably from about 0.1 mg. to about 10 mg. per kg. of body weight per day. The composition of the invention are preferably administered orally but parenteral routes such as subcutaneous, intramuscular, and intravenous can also be employed as can topical routes of administration. The daily dose can be administered in an amount from about 0.1 to about 500 mg, preferably from about 0.2 to about 100 mg singly or can be divided into two to four doses administered throughout the day.

The formulations as described above will be administered for a prolonged period, that is, for as long as the potential for high blood pressure and congestive heart failure and the other diseases set out hereinbefore, continue. Sustained release forms of such formulations which may provide such amounts daily, biweekly, weekly, monthly and the like may also be employed.

Combinations

In addition, in accordance with the present invention, the modified-release formulation of the invention may optionally include in addition to the vasopeptidase inhibitor any and all therapeutic agents which are useful in combination with vasopeptidase inhibitors and/or ACE inhibitors and/or NEP inhibitors.

Thus, where desired, the modified-release formulation of the invention may be used in combination with human ANF99-126, one or more diuretics, one or more antihypertensive agents, one or more platelet aggregation inhibitors, and/or one or more other cardiovascular agents (including anti-anginal agents, anti-arrhythmic agents, anti-atherosclerosis agents, anti-inflammatory agents, anti-heart failure agents), one or more hypolipidemic agents, one or more lipid-lowering agents, lipid agents, or lipid modulating agents, one or more antidiabetic agents, anti-obesity agents, one or more of the following therapeutic agents: anti-Alzheimer's agents, anti-dementia agents, anti-osteoporosis agents, hormone replacement agents, anti-cancer agents, anti-infective agents, growth hormone secretagogues, selective androgen receptor modulators, and/or other therapeutic agents which may be administered orally in the same dosage form or in a separate oral dosage form, or by injection.

Diuretics which may be employed in combination with the formulation of the invention include hydrochlorothiazide, torasemide, furosemide, dichlorophenamide, spironolactone, indapamide, polythiazide, methyclothiazide, chlorothiazide, hydroflumethiazide, ethacrynic acid, triamterene, amiloride, metolazone, chlorthalidone and mixtures of two or more of any of the above diuretics.

Preferred diuretics are hydrochlorothiazide and furosemide.

A preferred combination is omapatrilat or gemopatrilat, and hydrochlorothiazide or furosemide.

Most preferred is a bilayer tablet combination of immediate release hydrochlorothiazide layer and a modified-release matrix layer of omapatrilat. A compression-coated tablet may also be used where the core is a matrix tablet of omapatrilat.

Antiplatelet agents which may be employed in combination with the formulation of the invention include aspirin, clopidogrel, ticlopidine, dipyridamole, abciximab, tirofiban, eptifibatide, anagrelide, and ifetroban, with clopidogrel and aspirin being preferred.

The antihypertensive agents which may be employed in combination with the vasopeptidase inhibitor include ACE inhibitors, angiotensin II receptor antagonists, NEP inhibitors such as candoxatril, NEP/ACE inhibitors, as well as calcium channel blockers (such as verapamil and amlodipine besylate), T-channel calcium antagonists (such as mibefradil), β-adrenergic blockers, diuretics, α-adrenergic blockers (such as doxazosin mesylate and terazosin HCl), dual action receptor antagonists (DARA), heart failure drugs such as digoxin, and other types of antihypertensive agents.

The angiotensin converting enzyme inhibitor which may be employed herein includes those containing a mercapto (—S—) moiety such as substituted proline derivatives, such as any of those disclosed in U.S. Pat. No. 4,046,889 to Ondetti et al mentioned above, with captopril, that is, 1-[(2S)-3-mercapto-2-methylpropionyl]-L-proline, being preferred, and mercaptoacyl derivatives of substituted prolines such as any of those disclosed in U.S. Pat. No. 4,316,906 with zofenopril being preferred.

Other examples of mercapto containing ACE inhibitors that may be employed herein include rentiapril (fentiapril, Santen) disclosed in Clin. Exp. Pharmacol. Physiol. 10:131 (1983); as well as pivopril and YS980.

Other examples of angiotensin converting enzyme inhibitors which may be employed herein include any of those disclosed in U.S. Pat. No. 4,374,829 mentioned above, with N-(1-ethoxycarbonyl-3-phenylpropyl)-L-alanyl-L-proline, that is, enalapril, being preferred, any of the phosphonate substituted amino or imino acids or salts disclosed in U.S. Pat. No. 4,452,790 with (S)-1-[6-amino-2-[[hydroxy-(4-phenylbutyl)phosphinyl]oxy]-1-oxohexyl]-L-proline or (ceronapril) being preferred, phosphinylalkanoyl prolines disclosed in U.S. Pat. No. 4,168,267 mentioned above with fosinopril being preferred, any of the phosphinylalkanoyl substituted prolines disclosed in U.S. Pat. No. 4,337,201, and the phosphonamidates disclosed in U.S. Pat. No. 4,432,971 discussed above.

Other examples of ACE inhibitors that may be employed herein include Beecham's BRL 36,378 as disclosed in European Patent Application Nos. 80822 and 60668; Chugai's MC-838 disclosed in C. A. 102:72588v and Jap. J. Pharmacol. 40:373 (1986); Ciba-Geigy's CGS 14824 (3-([1-ethoxycarbonyl-3-phenyl-(1S)-propyl]amino)-2,3,4,5-tetrahydro-2-oxo-1-(3S)-benzazepine-1 acetic acid HCl) disclosed in U.K. Patent No. 2103614 and CGS 16,617 (3(S)-[[(1S)-5-amino-1-carboxypentyl]amino]-2,3,4,5-tetrahydro-2-oxo-1H-1-benzazepine-1-ethanoic acid) disclosed in U.S. Pat. No. 4,473,575; cetapril (alacepril, Dainippon) disclosed in Eur. Therap. Res. 39:671 (1986); 40:543 (1986); ramipril (Hoechsst) disclosed in Euro. Patent No. 79-022 and Curr. Ther. Res. 40:74 (1986); Ru 44570 (Hoechst) disclosed in Arzneimittelforschung 34:1254 (1985), cilazapril (Hoffman-LaRoche) disclosed in J. Cardiovasc. Pharmacol. 9:39 (1987); R 31-2201 (Hoffman-LaRoche) disclosed in FEBS Lett. 165:201 (1984); lisinopril (Merck), indalapril (delapril) disclosed in U.S. Pat. No. 4,385,051; indolapril (Schering) disclosed in J. Cardiovasc. Pharmacol. 5:643, 655 (1983), spirapril (Schering) disclosed in Acta. Pharmacol. Toxicol. 59 (Supp. 5):173 (1986); perindopril (Servier) disclosed in Eur. J. clin. Pharmacol. 31:519 (1987); quinapril (Warner-Lambert) disclosed in U.S. Pat. No. 4,344,949 and CI925 (Warner-Lambert) ([3S-[2[R(*)R(*)]]3R(*)]-2-[2-[[1-(ethoxy-carbonyl)-3-phenylpropyl]amino]-1-oxopropyl]-1,2,3,4-tetrahydro-6,7-dimethoxy-3-isoquinolinecarboxylic acid HCl)disclosed in Pharmacologist 26:243, 266 (1984), WY-44221 (Wyeth) disclosed in J. Med. Chem. 26:394 (1983).

Preferred ACE inhibitors are captopril, fosinopril, enalapril, lisinopril, quinapril, benazepril, fentiapril, ramipril and moexipril.

The angiotensin II receptor antagonist (also referred to herein as angiotensin II antagonist or AII antagonist) suitable for use herein includes, but is not limited to, irbesartan, losartan, valsartan, candesartan, tasosartan or eprosartan, with irbesartan, losartan or valsartan being preferred.

A preferred oral dosage form, such as tablets or capsules, will contain the ACE inhibitor or AII antagonist in an amount within the range from abut 0.1 to about 500 mg, preferably from about 5 to about 200 mg and more preferably from about 10 to about 150 mg.

For parenteral administration, the ACE inhibitor, angiotensin II antagonist or NEP/ACE inhibitor will be employed in an amount within the range from about 0.005 mg/kg to about 10 mg/kg and preferably from about 0.01 mg/kg to about 1 mg/kg.

Where a drug is to be administered intravenously, it will be formulated in conventional vehicles, such as distilled water, saline, Ringer's solution or other conventional carriers.

It will be appreciated that preferred dosages of ACE inhibitor and AII antagonist will be as set out in the latest edition of the Physician's Desk Reference (PDR).

Dual action receptor antagonists (DARA) suitable for use herein include those disclosed in U.S. applications Ser. No. 09/513,779, filed Feb. 25, 2000, and Ser. No. 09/604,322, filed Jun. 26, 2000.

Other examples of preferred antihypertensive agents suitable for use herein include amlodipine besylate (Norvasc®), prazosin HCl (Minipress®), verapamil, nifedipine, diltiazem, felodipine, nisoldipine, isradipine, nicardipine,, beta blockers such as nadolol, atenolol (Tenormin®), sotalol, terazosin, doxazosin, carvedilol, and propranolol, and clonidine HCl (Catapres®).

The antiarrhythmic agents suitable for use herein include β-blockers as set out herein including sotalol and amioderome, calcium channel blockers as set out herein including verapamil, nifedipine, amlodipine-besylate, and diltiazem, which may also be used in combination with a debrillator device such as a pace maker.

Anti-anginal agents such as vasodilators, suitable for use herein include for example, isosorbide dinitrate, or nitroglycerin.

The antihypertensive agents, diuretics and antiplatelet drugs and antiarrhythmics and antianginal agents may be employed in amounts as indicated in the PDR. Ifetroban may be employed in amounts as set out in U.S. Patent No. 5,100,889.

The hypolipidemic agent or lipid-lowering agent or other lipid agent or lipid modulating agent which may be optionally employed in the composition of the invention may include 1,2,3 or more MTP inhibitors, HMG CoA reductase inhibitors, squalene synthetase inhibitors, PPAR α agonists, PPAR dual αγ agonists, PPAR δ agonists, fibric acid derivatives, ACAT inhibitors, lipoxygenase inhibitors, cholesterol absorption inhibitors, ileal Na⁺/bile acid cotransporter inhibitors, upregulators of LDL receptor activity, cholesteryl ester transfer protein inhibitors, bile acid sequestrants, and/or nicotinic acid and derivatives thereof.

MTP inhibitors employed herein include MTP inhibitors disclosed in U.S. Pat. No. 5,595,872, U.S. Pat. No. 5,739,135, U.S. Pat. No. 5,712,279, U.S. Pat. No. 5,760,246, U.S. Pat. No. 5,827,875, U.S. Pat. No. 5,885,983 and U.S. application Ser. No. 09/175,180 filed Oct. 20, 1998, now U.S. Pat. No. 5,962,440. Preferred are each of the preferred MTP inhibitors disclosed in each of the above patents and applications.

All of the above U.S. patents and applications are incorporated herein by reference.

Most preferred MTP inhibitors to be employed in accordance with the present invention include preferred MTP inhibitors as set out in U.S. Pat. Nos. 5,739,135 and 5,712,279, and U.S. Pat. No. 5,760,246.

The hypolipidemic agent may be an HMG CoA reductase inhibitor which includes, but is not limited to, mevastatin and related compounds as disclosed in U.S. Pat. No. 3,983,140, lovastatin (mevinolin) and related compounds as disclosed in U.S. Pat. No. 4,231,938, pravastatin and related compounds such as disclosed in U.S. Pat. No. 4,346,227, simvastatin and related compounds as disclosed in U.S. Pat. Nos. 4,448,784 and 4,450,171 and compounds disclosed in U.S. application Ser. No. 09/875,155 filed Jun. 6, 2001, and U.S. application Ser. No. 09/875,218 filed Jun. 6, 2001. Other HMG CoA reductase inhibitors which may be employed herein include, but are not limited to, fluvastatin, disclosed in U.S. Pat. No. 5,354,772, cerivastatin disclosed in U.S. Pat. Nos. 5,006,530 and 5,177,080, atorvastatin disclosed in U.S. Pat. Nos. 4,681,893, 5,273,995, 5,385,929 and 5,686,104, pitavastatin (Nissan/Sankyo's nisvastatin (NK-104)) or itavastatin) disclosed in U.S. Pat. No. 5,011,930, Shionogi-Astra/Zeneca rosuvastatin (visastatin (ZD-4522) disclosed in U.S. Pat. No. 5,260,440, and related statin compounds disclosed in U.S. Pat. No. 5,753,675, pyrazole analogs of mevalonolactone derivatives as disclosed in U.S. Pat. No. 4,613,610, indene analogs of mevalonolactone derivatives as disclosed in PCT application WO 86/03488, 6-[2-(substituted-pyrrol-1-yl)-alkyl)pyran-2-ones and derivatives thereof as disclosed in U.S. Pat. No. 4,647,576, Searle's SC-45355 (a 3-substituted pentanedioic acid derivative) dichloroacetate, imidazole analogs of mevalonolactone as disclosed in PCT application WO 86/07054, 3-carboxy-2-hydroxy-propane-phosphonic acid derivatives as disclosed in French Patent No. 2,596,393, 2,3-disubstituted pyrrole, furan and thiophene derivatives as disclosed in European Patent Application No. 0221025, naphthyl analogs of mevalonolactone as disclosed in U.S. Pat. No. 4,686,237, octahydronaphthalenes such as disclosed in U.S. Pat. No. 4,499,289, keto analogs of mevinolin (lovastatin) as disclosed in European Patent Application No.0,142,146 A2, and quinoline and pyridine derivatives disclosed in U.S. Pat. Nos. 5,506,219 and 5,691,322.

In addition, phosphinic acid compounds useful in inhibiting HMG CoA reductase suitable for use herein are disclosed in GB 2205837.

The squalene synthetase inhibitors suitable for use herein include, but are not limited to, α-phosphono-sulfonates disclosed in U.S. Pat. No. 5,712,396, those disclosed by Biller et al, J. Med. Chem., 1988, Vol. 31, No. 10, pp 1869-1871, including isoprenoid (phosphinyl-methyl)phosphonates as well as other known squalene synthetase inhibitors, for example, as disclosed in U.S. Pat. No. 4,871,721 and 4,924,024 and in Biller, S. A., Neuenschwander, K., Ponpipom, M. M., and Poulter, C. D., Current Pharmaceutical Design, 2, 1-40 (1996).

In addition, other squalene synthetase inhibitors suitable for use herein include the terpenoid pyrophosphates disclosed by P. Ortiz de Montellano et al, J. Med. Chem., 1977, 20, 243-249, the farnesyl diphosphate analog A and presqualene pyrophosphate (PSQ-PP) analogs as disclosed by Corey and Volante, J. Am. Chem. Soc., 1976, 98, 1291-1293, phosphinylphosphonates reported by McClard, R. W. et al, J.A.C.S., 1987, 109, 5544 and cyclopropanes reported by Capson, T. L., PhD dissertation, June, 1987, Dept. Med. Chem. U of Utah, Abstract, Table of Contents, pp 16, 17, 40-43, 48-51, Summary.

Other hypolipidemic agents suitable for use herein include, but are not limited to, fibric acid derivatives, such as fenofibrate, gemfibrozil, clofibrate, bezafibrate, ciprofibrate, clinofibrate and the like, probucol, and related compounds as disclosed in U.S. Patent No. 3,674,836, probucol and gemfibrozil being preferred, bile acid sequestrants such as cholestyramine, colestipol and DEAE-Sephadex (Secholex®, Policexide®) and cholestagel (Sankyo/Geltex), as well as lipostabil (Rhone-Poulenc), Eisai E-5050 (an N-substituted ethanolamine derivative), imanixil (HOE-402), tetrahydrolipstatin (THL), istigmastanylphos-phorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid (niacin), acipimox, acifran, neomycin, p-aminosalicylic acid, aspirin, poly(diallylmethylamine) derivatives such as disclosed in U.S. Pat. No. 4,759,923, quaternary amine poly(diallyldimethylammonium chloride) and ionenes such as disclosed in U.S. Pat. No. 4,027,009, and other known serum cholesterol lowering agents.

The hypolipidemic agent may be an ACAT inhibitor (which also has anti-atherosclerosis activity) such as disclosed in, Drugs of the Future 24, 9-15 (1999), (Avasimibe); “The ACAT inhibitor, C1-1011 is effective in the prevention and regression of aortic fatty streak area in hamsters”, Nicolosi et al, Atherosclerosis (Shannon, Irel). (1998), 137(1), 77-85; “The pharmacological profile of FCE 27677: a novel ACAT inhibitor with potent hypolipidemic activity mediated by selective suppression of the hepatic secretion of ApoB100-containing lipoprotein”, Ghiselli, Giancarlo, Cardiovasc. Drug Rev. (1998), 16(1), 16-30; “RP 73163: a bioavailable alkylsulfinyl-diphenylimidazole ACAT inhibitor”, Smith, C., et al, Bioorg. Med. Chem. Lett. (1996), 6(1), 47-50; “ACAT inhibitors: physiologic mechanisms for hypolipidemic and anti-atherosclerotic activities in experimental animals”, Krause et al, Editor(s): Ruffolo, Robert R., Jr.; Hollinger, Mannfred A., Inflammation: Mediators Pathways (1995), 173-98, Publisher: CRC, Boca Raton, Fla.; “ACAT inhibitors: potential anti-atherosclerotic agents”, Sliskovic et al, Curr. Med. Chem. (1994), 1(3), 204-25; “Inhibitors of acyl-CoA:cholesterol O-acyl transferase (ACAT) as hypocholesterolemic agents. 6. The first water-soluble ACAT inhibitor with lipid-regulating activity. Inhibitors of acyl-CoA:cholesterol acyltransferase (ACAT). 7. Development of a series of substituted N-phenyl-N′-[(1-phenylcyclopentyl)methyl]ureas with enhanced hypocholesterolemic activity”, Stout et al, Chemtracts: Org. Chem. (1995), 8(6), 359-62, or TS-962 (Taisho Pharmaceutical Co. Ltd) as well as F-1394, CS-505, F-12511, HL-004, K10085 and YIC-C8-434.

The hypolipidemic agent may be an upregulator of LD2 receptor activity such as MD-700 (Taisho Pharmaceutical Co. Ltd) and LY295427 (Eli Lilly).

The hypolipidemic agent may be a cholesterol absorption inhibitor preferably Schering-Plough's SCH48461 (ezetimibe) as well as those disclosed in Atherosclerosis 115, 45-63 (1995) and J. Med. Chem. 41, 973 (1998).

The lipid agent or lipid-modulating agent may be a cholesteryl transfer protein inhibitor (CETP) such as Pfizer's CP-529,414 as well as those disclosed in WO/0038722 and in EP 818448 (Bayer) and EP 992496, and Pharmacias SC-744 and SC-795, as well as CETi-1 and JTT-705.

The hypolipidemic agent may be an ileal Na⁺/bile acid cotransporter inhibitor such as disclosed in Drugs of the Future, 24, 425-430 (1999).

The ATP citrate lyase inhibitor which may be employed in the composition of the invention may include, for example, those disclosed in U.S. Pat. No. 5,447,954.

The lipid agent also includes a phytoestrogen compound such as disclosed in WO 00/30665 including isolated soy bean protein, soy protein concentrate or soy flour as well as an isoflavone such as genistein, daidzein, glycitein or equol, or phytosterols, phytostanol or tocotrienol as disclosed in WO 2000/015201;

a beta-lactam cholesterol absorption inhibitor such as disclosed in EP 675714;

an HDL upregulator such as an LXR agonist, a PPAR α-agonist and/or an FXR agonist;

an α-glucosidase inhibitor, an aldose reductase inhibitor and/or an LDL catabolism promoter such as disclosed in EP 1022272;

a sodium-proton exchange inhibitor such as disclosed in DE 19622222;

an LDL-receptor inducer or a steroidal glycoside such as disclosed in U.S. Pat. No. 5,698,527 and GB 2304106;

an anti-oxidant such as beta-carotene, ascorbic acid, α-tocopherol or retinol as disclosed in WO 94/15592 as well as Vitamin C and an antihomocysteine agent such as folic acid, a folate, Vitamin B6, Vitamin B12 and Vitamin E;

isoniazid as disclosed in WO 97/35576;

a cholesterol absorption inhibitor, an HMG-COA synthase inhibitor, or a lanosterol demethylase inhibitor as disclosed in WO 97/48701;

a PPAR δ agonist for treating dyslipidemia;

or a sterol regulating element binding protein-I (SREBP-1) as disclosed in WO 2000/050574, for example, a sphingolipid, such as ceramide, or neutral sphingomyelenase (N-SMase) or fragment thereof.

Preferred hypolipidemic agents are pravastatin, lovastatin, simvastatin, atorvastatin, fluvastatin, cerivastatin, pitavastatin and rosuvastatin, as well as niacin and/or cholestagel.

The above-mentioned U.S. patents are incorporated herein by reference. The amounts and dosages employed will be as indicated in the Physician's Desk Reference and/or in the patents set out above or as otherwise known in the art.

The vasopeptidase inhibitor present in the composition of the invention will be employed in a weight ratio to the hypolipidemic agent (were present), within the range from about 500:1 to about 1:500, preferably from about 100:1 to about 1:100.

The dose administered must be carefully adjusted according to age, weight and condition of the patient, as well as the route of administration, dosage form and regimen and the desired result.

The dosages and formulations for the hypolipidemic agent or lipid agent or lipid modulating agent will be as disclosed in the various patents and applications discussed above.

The dosages and formulations for the hypolipidemic agent or lipid agent or lipid modulating agent to be employed, where applicable, will be as set out in the latest edition of the Physicians' Desk Reference.

For oral administration, a satisfactory result may be obtained employing the MTP inhibitor in an amount within the range of from about 0.01 mg to about 500 mg and preferably from about 0.1 mg to about 100 mg, one to four times daily.

A preferred oral dosage form, such as tablets or capsules, will contain the MTP inhibitor in an amount of from about 1 to about 500 mg, preferably from about 2 to about 400 mg, and more preferably from about 5 to about 250 mg, one to four times daily.

For oral administration, a satisfactory result may be obtained employing an HMG CoA reductase inhibitor, for example, pravastatin, lovastatin, simvastatin, atorvastatin, fluvastatin or cerivastatin in dosages employed as indicated in the Physician's Desk Reference, such as in an amount within the range of from about 1 to 2000 mg, and preferably from about 4 to about 200 mg.

The squalene synthetase inhibitor may be employed in dosages in an amount within the range of from about 10 mg to about 2000 mg and preferably from about 25 mg to about 200 mg.

A preferred oral dosage form, such as tablets or capsules, will contain the HMG CoA reductase inhibitor in an amount from about 0.1 to about 100 mg, preferably from about 0.5 to about 80 mg, and more preferably from about 1 to about 40 mg.

A preferred oral dosage form, such as tablets or capsules will contain the squalene synthetase inhibitor in an amount of from about 10 to about 500 mg, preferably from about 25 to about 200 mg.

The anti-atherosclerotic agent includes a lipoxygenase inhibitor including a 15-lipoxygenase (15-LO) inhibitor such as benzimidazole derivatives as disclosed in WO 97/12615, 15-LO inhibitors as disclosed in WO 97/12613, isothiazolones as disclosed in WO 96/38144, and 15-LO inhibitors as disclosed by Sendobry et al “Attenuation of diet-induced atherosclerosis in rabbits with a highly selective 15-lipoxygenase inhibitor lacking significant antioxidant properties,” Brit. J. Pharmacology (1997) 120, 1199-1206, and Cornicelli et al, “15-Lipoxygenase and its Inhibition: A Novel Therapeutic Target for Vascular Disease”, Current Pharmaceutical Design, 1999, 5, 11-20.

The vasopeptidase inhibitor and the hypolipidemic agent may be employed together in the same oral dosage form or in separate oral dosage forms taken at the same time.

The compositions described above may be administered in the dosage forms as described above in single or divided doses of one to four times daily. It may be advisable to start a patient on a low dose combination and work up gradually to a high dose combination.

The antidiabetic agent which may be optionally employed in combination with the vasopeptidase inhibitor may be 1,2,3 or more antidiabetic agents or antihyperglycemic agents including insulin secretagogues or insulin sensitizers, which may include biguanides, sulfonylureas, glucosidase inhibitors, aldose reductase inhibitors, PPAR γ agonists such as thiazolidinediones, PPAR α agonists (such as fibric acid derivatives), PPAR δ antagonists or agonists, aP2 inhibitors, PPAR α/γ dual agonists, dipeptidyl peptidase IV (DP4) inhibitors, SGLT2 inhibitors, glycogen phosphorylase inhibitors, and/or meglitinides, as well as insulin, and/or glucagon-like peptide-1 (GLP-1), and/or a PTP-1B inhibitor (protein tyrosine phosphatase-1B inhibitor).

The antidiabetic agent may be an oral antihyperglycemic agent preferably a biguanide such as metformin or salts thereof, preferably metformin HCl.

Where the antidiabetic agent is a biguanide, the compounds of structure I will be employed in a weight ratio to biguanide within the range from about 0.001:1 to about 10:1, preferably from about 0.01:1 to about 5:1.

The antidiabetic agent may also preferably be a sulfonylurea such as glyburide (also known as glibenclamide), glimepiride (disclosed in U.S. Pat. No. 4,379,785), glipizide, gliclazide or chlorpropamide, other known sulfonylureas or other antihyperglycemic agents which act on the ATP-dependent channel of the β-cells, with glyburide and glipizide being preferred, which may be administered in the same or in separate oral dosage forms.

The vasopeptidase inhibitor will be employed in a weight ratio to the sulfonyl urea in the range from about 0.01:1 to about 100:1, preferably from about 0.02:1 to about 5:1.

The oral antidiabetic agent may also be a glucosidase inhibitor such as acarbose (disclosed in U.S. Pat. No. 4,904,769) or miglitol (disclosed in U.S. Pat. No. 4,639,436), which may be administered in the same or in a separate oral dosage forms.

The vasopeptidase inhibitor will be employed in a weight ratio to the glucosidase inhibitor within the range from about 0.01:1 to about 100:1, preferably from about 0.05:1 to about 10:1.

The vasopeptidase inhibitor may be employed in combination with a PPAR y agonist such as a thiazolidinedione oral anti-diabetic agent or other insulin sensitizers (which has an insulin sensitivity effect in NIDDM patients) such as troglitazone (Warner-Lambert's Rezulin®, disclosed in U.S. Pat. No. 4,572,912), rosiglitazone (SKB), pioglitazone (Takeda), Mitsubishi's MCC-555 (disclosed in U.S. Pat. No. 5,594,016), Glaxo-Welcome's GL-262570, englitazone (CP-68722, Pfizer) or darglitazone (CP-86325, Pfizer, isaglitazone (MIT/J&J), JTT-501 (JPNT/P&U), L-895645 (Merck), R-119702 (Sankyo/WL), NN-2344 (Dr. Reddy/NN), or YM-440 (Yamanouchi), preferably rosiglitazone and pioglitazone.

The vasopeptidase inhibitor will be employed in a weight ratio to the thiazolidinedione in an amount within the range from about 0.01:1 to about 100:1, preferably from about 0.05:1 to about 10:1.

The sulfonyl urea and PPAR y agonists in amounts of less than about 150 mg oral antidiabetic agent may be incorporated in a single tablet with the vasopeptidase inhibitor.

The vasopeptidase inhibitor may also be employed in combination with a antihyperglycemic agent such as insulin or with glucagon-like peptide-1 (GLP-1) or mimetic such as GLP-1(1-36) amide, GLP-1(7-36) amide, GLP-1(7-37) (as disclosed in U.S. Pat. No. 5,614,492 to Habener, the disclosure of which is incorporated herein by reference), as well as AC2993 (Amylen) and LY-315902 (Lilly), which may be administered via injection, intranasal, inhalation or by transdermal or buccal devices.

Where present, metformin, the sulfonyl ureas, such as glyburide, glimepiride, glipyride, glipizide, chlorpropamide and gliclazide and the glucosidase inhibitors acarbose or miglitol or insulin (injectable, pulmonary, buccal, or oral) may be employed in formulations as described above and in amounts and dosing as indicated in the Physician's Desk Reference (PDR).

Where present, metformin or salt thereof may be employed in amounts within the range from about 500 to about 2000 mg per day which may be administered in single or divided doses one to four times daily.

Where present, the PPAR anti-diabetic agent may be employed in amounts within the range from about 0.01 to about 2000 mg/day which may be administered in single or divided doses one to four times per day.

Where present insulin and other anti-diabetic agents as set out above may be employed in formulations, amounts and dosing as indicated by the Physician's Desk Reference.

Where present GLP-1 peptides or mimetics may be administered in oral buccal formulations, by nasal administration or parenterally as described in U.S. Pat. Nos. 5,346,701 (TheraTech), 5,614,492 and 5,631,224 which are incorporated herein by reference.

The antidiabetic agent or other lipid agent may also be a PPAR modulator such as a PPAR α/γ dual agonist such as AR-HO39242 (Astra/Zeneca), GW-409544 (Glaxo-Wellcome), KRP297 (Kyorin Merck) as well as those disclosed by Murakami et al, “A Novel Insulin Sensitizer Acts As a Coligand for Peroxisome Proliferation-Activated Receptor Alpha (PPAR alpha) and PPAR gamma. Effect on PPAR alpha Activation on Abnormal Lipid Metabolism in Liver of Zucker Fatty Rats”, Diabetes 47, 1841-1847 (1998), and in U.S. application Ser. No. 09/664,598, filed Sep. 18, 2000, (attorney file LA29), provisional applications Ser. No. 60/294,505 and 60/294,380, each filed May 30, 2001 (attorney files LA72 and LA73), the disclosures of which are incorporated herein by reference, employing dosages as set out therein, which compounds designated as preferred are preferred for use herein.

The antidiabetic agent may be an SGLT2 inhibitor such as disclosed in U.S. application Ser. No. 09/679,027, filed Oct. 4, 2000 (attorney file LA49), employing dosages as set out therein. Preferred are the compounds designated as preferred in the above application.

The antidiabetic agent may be an aP2 inhibitor such as disclosed in U.S. application Ser. No. 09/391,053, filed Sep. 7, 1999, and in U.S. application Ser. No. 09/519,079, filed Mar. 6, 2000 (attorney file LA27), employing dosages as set out herein. Preferred are the compounds designated as preferred in the above application.

The antidiabetic agent may be a DP4 inhibitor such as disclosed in application Ser. No. 09/788,173, filed Feb. 16, 2001 (attorney file LA50), WO99/38501, WO99/46272, WO99/67279 (PROBIODRUG), WO99/67278 (PROBIODRUG), WO99/61431 (PROBIODRUG), NVP-DPP728A (1-[[[2-[(5-cyanopyridin-2-yl)amino]ethyl]amino]acetyl]-2-cyano-(S)-pyrrolidine) (Novartis) (preferred) as disclosed by Hughes et al, Biochemistry, 38(36), 11597-11603, 1999, TSL-225 (tryptophyl-1,2,3,4-tetrahydro-isoquinoline-3-carboxylic acid (disclosed by Yamada et al, Bioorg. & Med. Chem. Lett. 8 (1998) 1537-1540, 2-cyanopyrrolidides and 4-cyanopyrrolidides as disclosed by Ashworth et al, Bioorg. & Med. Chem. Lett., Vol. 6, No. 22, pp 1163-1166 and 2745-2748 (1996) employing dosages as set out in the above references.

The meglitinide which may optionally be employed in combination with vasopeptidase inhibitor may be repaglinide or Starlix® (Novartis), nateglinide (Novartis) or KAD1229 (PF/Kissei), with repaglinide being preferred.

The antidiabetic compound may be a melanocortin receptor agonist such as a spiropiperidine as disclosed in WO 99/64002.

The vasopeptidase inhibitor will be employed in a weight ratio to the meglitinide, PPAR modulator such as a PPAR γ agonist, PPAR γ antagonist, PPAR α agonist, PPAR δ agents or antagonist, PPAR α/γ dual agonist, aP2 inhibitor, DP4 inhibitor or SGLT2 inhibitor or other antidiabetic agent within the range from about 0.01:1 to about 100:1, preferably from about 0.05:1 to about 10:1.

The other type of therapeutic agent which may be optionally employed with the vasopeptidase inhibitor may be 1, 2, 3 or more of an anti-obesity agent including a beta 3 adrenergic agonist, a lipase inhibitor, a serotonin (and dopamine) reuptake inhibitor, an aP2 inhibitor, a thyroid receptor beta drug, a PTP-1B inhibitor, an anorectic agent, a PPAR modulator including PPAR γ antagonists, PPAR α agonists, PPAR δ antagonists, a CCKA agonist, a leptin inhibitor such as a leptin receptor activator, a neuropeptide Y antagonist, a melanocortin-4-receptor (MC4R) agonist, a fatty acid oxidation upregulator or inducer (such as Famoxin® Genset).

The beta 3 adrenergic agonist which may be optionally employed in combination with the vasopeptidase inhibitor may be AJ9677 (Takeda/Dainippon), L750355 (Merck), or CP331648 (Pfizer) or other known beta 3 agonists as disclosed in U.S. Pat. Nos. 5,541,204, 5,770,615, 5,491,134, 5,776,983 and 5,488,064, with AJ9677, L750,355 and CP331648 being preferred.

The neuropeptide Y antagonists which may be optionally employed in combination with the vasopeptidase inhibitor include those described in WO 0113917 (BMS) or in U.S. Pat. No. 6,218,408 (Synaptic) and in WO 0114376 (Banyu).

The lipase inhibitor which may be optionally employed in combination with the vasopeptidase inhibitor may be orlistat or ATL-962 (Alizyme), with orlistat being preferred.

The serotonin (and dopoamine) reuptake inhibitor which may be optionally employed in combination with a compound of formula I may be sibutramine, topiramate (Johnson & Johnson) or axokine (Regeneron), with sibutramine and topiramate being preferred.

The thyroid receptor beta compound which may be optionally employed in combination with the vasopeptidase inhibitor may be a thyroid receptor ligand as disclosed in WO97/21993 (U. Cal SF), WO99/00353 (KaroBio), GB98/284425 (KaroBio), and U.S. Provisional Application 60/183,223 filed Feb. 17, 2000, with compounds of the KaroBio applications and the above U.S. provisional application being preferred.

The anorectic agent which may be optionally employed in combination with the vasopeptidase inhibitor may be dexamphetamine, phentermine, phenylpropanolamine or mazindol, with dexamphetamine being preferred.

The CCKA agonists which may be employed herein include Glaxo-SmithKline's GI-181,771 and Sanofi's SR146,131.

The PTP-1B inhibitor which may be an anti-obesity and/or an antidiabetic agent include those disclosed in WO 99/585,521, WO 99/58518, WO 99/58522 and WO 99/61435.

The anti-obesity agent employed may also be Pfizer's P57 or CP-644,673 (licensed from Phytopharm).

The various anti-obesity agents described above may be employed in the same dosage form with the composition of the invention in different dosage forms, in dosages and regimens as generally known in the art or in the PDR.

Anti-Alzheimer's agents or anti-dementia agents suitable for use herein with the formulation of the invention include tacrine HCl (Cognex®) and donepezil (Aricept®), as well as γ-secretase inhibitors, β-secretase inhibitors and/or antihypertensive agents. Dosages employed will be as set out in the PDR.

Antiosteoporosis agents suitable for use herein in combination with the formulation of the invention include parathyroid hormone or bisphosphonates, such as MK-217 (alendronate) (Fosamax®) as well as Ca receptor agonists and progestin receptor agonists. Dosages employed will be as set out in the PDR.

The hormone replacement therapeutic agents, where present, will be employed in dosages as set out in the latest edition of the PDR. Examples of such agents include selective estrogen receptor modulators (SERMs) such as raloxifen, tamoxifen or lasoxifen.

The formulation of the invention may also be employed in combination with a tyrosine kinase inhibitor such as disclosed in WO 2000/053605;

the selective androgen receptor modulator suitable for use herein may be LGD-2226 (Ligand);

coenzyme Q sub. 10 such as disclosed in U.S. Pat. Nos. 5,316,765, 4,933,165, 4,929,437;

an agent that upregulates type III endothelial cell nitric acid syntase such as disclosed in WO 2000/003746;

a chondroprotective compound such as a polysulfated glycosaminoglycan (PSGAG), glucosamine, chondroitin sulfate (CS), hyaluronic acid (HA), pentosan polysulfate (PPS), doxycycline or minocycline, such as disclosed in EP 970694;

a cyclooxygenase (COX)-2 inhibitor, such as celecoxib (Celebrex® (Searle)) or rofecoxib (Vioxx® (Merck)) or a glycoprotein IIa/IIIb receptor antagonist such as disclosed in WO 99/45913 and tirofiban or abciximab;

a 5-HT reuptake inhibitor such as disclosed in WO 99/44609;

a growth hormone secretagogue such as disclosed in U.S. applications Ser. No. 09/662,448, filed Sep. 14, 2000, and U.S. Provisional application 60/203,335, filed May 11, 2000, and MK-677 (Merck), Pfizer's CP-424391 and Lilly's LY 444,711;

anti-atherosclerosis agents such as ACAT inhibitors and lipoxygenase inhibitors as described herein and phospholipase A-2 inhibitors such as S-3013 and SB-435,495 (which are also anti-inflammatory agents);

anti-infective agents such as quinolones, for example, ciprofloxacin, ofloxacin, and Tequin® (Bristol-Myers Squibb), macrolides such as erythromycin and clarithromycin (Biaxin® (Abbott)), and azithromycin (Zithromax (Pfizer)); or

an immunosuppressant (for use in transplantations) such as cyclosporine, mycophenolate mofetil, azathioprine and the like.

As used herein, the phrase “antineoplastic agent” refers to compounds which prevent cancer cells from multiplying. In general, the antineoplastic agents used herein prevent cancer cells from multiplying by: (1) interfering with the cell's ability to replicate DNA, or (2) inducing apoptosis in the cancerous cells.

Examples of antineoplastic agents which are suitable for use in combinations of this invention include, but are not limited to, microtuble-stabilizing agents such as the taxanes, for example, paclitaxel (also known as Taxol®), docetaxel (also known as Taxotere®), 7-O-methylthio-methylpaclitaxel (disclosed in U.S. Pat. No. 5,646,176), 3′-tert-butyl-3′-N-tert-butyloxycarbonyl-4-deacetyl-3′-dephenyl-3′-N-debenzoyl-4-O-methoxycarbonyl-paclitaxel (disclosed in U.S. Ser. No. 60/179,965 filed on Feb. 3, 2000, and example 17 herein), C-4 methyl carbonate paclitaxel (disclosed in WO 94/14787), the epothilone, such as epothilone A, epothilone B, epothilone C, epothilone D, desoxyepothilone A, desoxyepothilone B, [1S-[1R*,3R*(E),7R*,10S*,11R*,12R*,16S*]]-7,11-dihydroxy-8,8,10,12,16-pentamethyl-3-[l-methyl-2-(2-methyl-4-thiazolyl)ethenyl]-4-aza-17-oxabicyclo[14.1.0]hepta-decane-5,9-dione (disclosed in WO 99/02514), [1S-[1R*,3R*(E),7R*,10S*,11R*,12R*,16S*]]-3-[2-[2-(aminomethyl)-4-thiazolyl]-1-methylethenyl]-7,11-di-hydroxy-8,8,10,12,16-pentamethyl-4,17-dioxabi-cyclo[14.1.0]-heptadecane-5,9-dione (disclosed in U.S. Ser. No. 09/506,481 filed on Feb. 17, 2000, and examples 7 and 8 herein), and derivatives thereof; microtuble-disruptor agents; alkylating agents; anti-metabolites; epidophyllotoxin; an antineoplastic enzyme; a topoisomerase inhibitor; procarbazine; mitoxantrone; platinum coordination complexes; biological response modifiers; growth inhibitors; hormonal/antihormonal therapeutic agents; and haematopoietic growth factors.

Other classes of antineoplastic agents suitable for use with the formulation of the present invention include, but are not limited to, the anthracycline family of drugs, the vinca drugs, the mitomycins, the bleomycins, the cytotoxic nucleosides, discodermolide, the pteridine family of drugs, diynenes, aromatase inhibitors, and the podophyllotoxins. Particularly useful members of those classes not previously mentioned include, for example, doxorubicin, carminomycin, daunorubicin, aminopterin, methotrexate, methopterin, dichloro-methotrexate, mitomycin C, porfiromycin, 5-fluorouracil, 6-mercaptopurine, gemcitabine, cytosine arabinoside, podophyllotoxin or podophyllotoxin derivatives such as etoposide, etoposide phosphate or teniposide, melphalan, vinblastine, vincristine, leurosidine, vindesine, leurosine, and the like. Other useful antineoplastic agents include estramustine, cisplatin, carboplatin, cyclophosphamide, bleomycin, tamoxifen, ifosfamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons, and interleukins.

It will be appreciated that unless otherwise specified the dosage regiment for therapeutic agents used in combination with the compositions of the invention will be as specified in the PDR.

The following Examples represent preferred oral modified-release drug delivery systems for vasopeptidase inhibitors in accordance with the invention. However, alternate routes of administration (buccal, sublingual, nasal, transdermal, vaginal, rectal) can also be used for this invention. Other available modified-release delivery mechanisms may be employed, including but not limited to, diffusion controlled matrix systems, erodible systems, osmotic systems, and barrier membrane systems, transdermal systems, inhalation systems and buccal systems.

### EXAMPLE 1

Modified-release (MR-B) tablets of the invention were prepared from the following formulation:

| Ingredient | Mg/Tablet |
|---|---|
| Omapatrilat | 20.0 |
| Anhydrous Lactose | 93.0 |
| Microcrystalline Cellulose | 30.0 |
| Hydroxypropyl methylcellulose | 30.0 |
| (Methocel K100LV) | |
| Hydroxypropyl methylcellulose | 20.0 |
| (Methocel K4M) | |
| Stearic Acid | 6.0 |
| Silicon Dioxide | 1.0 |
| Tablet Weight | 200.0 |

Method of Manufacture: Omapatrilat, anhydrous lactose, hydroxypropyl celluloses, microcrystalline cellulose and a portion (17% of total) of stearic acid and (50% of total) silicon dioxide were mixed in a suitable blender. The blend was dry granulated using a roller compactor. The compacted ribbons were reduced to form granules (25 g to 1 mm particle size) using an appropriate screening device. The granules were mixed with the remaining portions of stearic acid and silicon dioxide in a suitable mixer. The final blend was compressed into tablets using a rotary tablet press.

The so-prepared modified-release omapatrilat tablets were found to deliver omapatrilat in a balanced manner to desired sites of absorption without undesirable surges or peaks in NEP or ACE inhibitory activity over the desired dosing interval.

### EXAMPLE 2

Modified-release (MR-C) tablets of the invention were prepared from the following formulation:

| Ingredient | Mg/Tablet |
|---|---|
| Omapatrilat | 20.0 |
| Anhydrous Lactose | 83.0 |
| Microcrystalline Cellulose | 30.0 |
| Hydroxypropyl methylcellulose | 60.0 |
| (Methocel K4M) | |
| Stearic Acid | 6.0 |
| Silicon Dioxide | 1.0 |
| Tablet Weight | 200.0 |

Method of Manufacture: Omapatrilat, anhydrous lactose, hydroxypropyl cellulose, microcrystalline cellulose and a portion of (17% of total) stearic acid and (50% of total) silicon dioxide were mixed in a suitable blender. The blend was dry granulated using a roller compactor. The compacted ribbons were reduced to form granules (25 μm to 1 mm particle size) using an appropriate screening device. The granules were mixed with the remaining portions of stearic acid and silicon dioxide in a suitable mixer. The final blend was compressed into tablets using a rotary tablet press.

The so-prepared modified-release omapatrilat tablets were found to deliver omapatrilat in a balanced manner to desired sites of absorption without undesirable surges or peaks in NEP or ACE inhibitory activity over the desired dosing interval.

### EXAMPLE 3

Modified-release coated beadlets of the invention were prepared from the following:

| Ingredient | Mg/Capsule |
|---|---|
| Core Beads | |
| Gemopatrilat | 30.0 |
| Microcrystalline Cellulose | 90.0 |
| Seal Coat | 3.6 |
| Hydroxypropyl cellulose | |
| Modified-Release Coat | |
| Eudragit RL-30D | 3.5 |
| Eudragit RS-30D | 3.5 |
| Triethyl Citrate | 1.2 |
| Talc | 6.0 |
| Encapsulation | |
| Beadlets | 137.8 |
| Hard Gelatin Capsule | 1 capsule |

Method of Manufacture: The core ingredients were thoroughly mixed and thereafter kneaded in a mixer with sufficient water to form a wet mass. The wet mass was passed through an extruder to form an extrudate with approximately 0.8 mm diameter. The extrudate was then passed through a spheronizer to form beadlets that were dried in a fluid bed dryer. The dried beadlets were then treated to form a seal coating by spraying with an aqueous solution of hydroxypropyl methylcellulose in a fluid bead coater. The beadlets were then coated with a modified-release coating comprising an aqueous solution of Eudragit RL-30D, Eudragit RS-30D, talc and triethyl citrate as the plasticzer. The beadlets were coated in a fluid bed coater. After coating the beads were cured in an oven. The cured beadlets were filled into gelatin capsules.

The so-prepared gemopatrilat modified-release beadlets were found to deliver gemopatrilat in a balanced manner to desired sites of absorption without undesirable surges or peaks in NEP or ACE inhibitory activity over the desired dosing interval.

### EXAMPLE 4

Modified-release tablets of the invention were prepared from the following formulation.

| Ingredient | Mg/Tablet |
|---|---|
| Gemopatrilat | 30.0 |
| Anhydrous Lactose | 139.5 |
| Microcrystalline Cellulose | 45.0 |
| Hydroxypropyl methylcellulose | 45.0 |
| (Methocel K100LV) | |
| Hydroxypropyl methylcellulose | 30.0 |
| (Methocel K4M) | |
| Stearic Acid | 9.0 |
| Silicon Dioxide | 1.5 |
| Tablet Weight | 300.0 |

Method of Manufacture: Gemopatrilat, anhydrous lactose, hydroxypropyl celluloses, microcrystalline cellulose and a portion of stearic acid (17% of total) and silicon dioxide (50% of total) were mixed in a suitable blender. The blend was dry granulated using a roller compactor. The compacted ribbons were reduced to form granules (25 μm to 1 mm particle size) using an appropriate screening device. The granules were mixed with the remaining portions of stearic acid and silicon dioxide in a suitable mixer. The final blend was compressed into tablets using a rotary tablet press.

The so-prepared gemopatrilat modified-release tablets were found to deliver gemopatrilat in a balanced manner to desired sites of absorption without undesirable surges or peaks in NEP or ACE inhibitory activity over the desired dosing interval.

### EXAMPLE 5

Modified-release tablets of the invention were prepared from the following formulation.

| Ingredient | Mg/Tablet |
|---|---|
| Gemopatrilat | 30.0 |
| Anhydrous Lactose | 124.5 |
| Microcrystalline Cellulose | 45.0 |
| Hydroxypropyl methylcellulose | 90.0 |
| (Methocel K4M) | |
| Stearic Acid | 9.0 |
| Silicon Dioxide | 1.5 |
| Tablet Weight | 300.0 |

Method of Manufacture: Gemopatrilat, anhydrous lactose, hydroxypropyl cellulose, microcrystalline cellulose and a portion of stearic acid (17% of total) and silicon dioxide (50% of total) were mixed in a suitable blender. The blend was dry granulated using a roller compactor. The compacted ribbons were reduced to form granules (25 μm to 1 mm particle size) using an appropriate screening device. The granules were mixed with the remaining portions of stearic acid and silicon dioxide in a suitable mixer. The final blend was compressed into tablets using a rotary tablet press.

The so-prepared gemopatrilat modified-release tablets were found to deliver gemopatrilat in a balanced manner to desired sites of absorption without undesirable surges or peaks in NEP or ACE inhibitory activity over the desired dosing interval.

Preparation of Immediate-Release Formulations (Controls A and B)

A. Immediate-release 10 mg omapatrilat Control A tablets were prepared from the following formulation as follows.

| Ingredient | Mg/Tablet |
|---|---|
| Omapatrilat | 10.0 |
| Anhydrous Lactose | 101.0 |
| Microcrystalline Cellulose | 78.0 |
| Croscarmellose Sodium | 6.0 |
| Stearic Acid | 5.0 |
| Tablet Weight | 200.0 |

Method of Manufacture: Omapatrilat, anhydrous lactose, microcrystalline cellulose, a portion of croscarmellose sodium (50% of total) and a portion of stearic acid (50% of total) were mixed in a suitable blender. The blend was dry granulated using a roller compactor. The compacted ribbons were reduced to form granules (25 μm to 1 mm particle size) using an appropriate screening device. The granules were mixed with the remaining portions of stearic acid and croscarmellose sodium in a suitable mixer. The final blend was compressed into tablets using a rotary tablet press.

B. Immediate-release 30 mg gemopatrilat Control B tablets were prepared from the following formulation as follows.

| Ingredient | Mg/Tablet |
|---|---|
| Gemopatrilat | 30.0 |
| Anhydrous Lactose | 89.0 |
| Microcrystalline Cellulose | 70.0 |
| Croscarmellose Sodium | 6.0 |
| Stearic Acid | 5.0 |
| Tablet Weight | 200.0 |

Method of Manufacture: Gemopatrilat, anhydrous lactose, microcrystalline cellulose, a portion of croscarmellose sodium (50% of total) and a portion of stearic acid (50% of total) were mixed in a suitable blender. The blend was dry granulated using a roller compactor. The compacted ribbons were reduced to form granules (25 μm to 1 mm particle size) using an appropriate screening device. The granules were mixed with the remaining portions of stearic acid and croscarmellose sodium in a suitable mixer. The final blend was compressed into tablets using a rotary tablet press.

### EXAMPLE 6

Experiment Comparing Modified-Release (MR) Formulations of the Invention (Examples 1 and 2 20 mg Omapatrilat Tablets) and Immediate-Release (IR) 10 mg Omapatrilat Control A Tablets

An open-label, single-dose, randomized, 3-period, 3-treatment, crossover study was performed in 24 subjects to evaluate the oral pharmacokinetic (PK) and phamacodynamic (PD) performance of the Example 1 modified-release 20 mg omapatrilat tablets (having a relatively fast onset), and the Example 2 modified-release 20 mg omapatrilat tablets (having a relatively slow onset), each relative to the immediate-release 10 mg omapatrilat Control A tablets.

Plasma samples were measured for unchanged omapatrilat using a validated LC/MS/MS method, as described below, and PK characterized using a non-compartmental method, as described below.

Bioanalytical:

Each blood sample was collected using Vacutainers® that contained K3EDTA as an anticoagulant and 50 μL of methyl acrylate as a derivatizing agent for the stabilization of omapatrilat and its thiol-containing metabolites that may be present in blood. Immediately after collection of the blood sample, the tube was gently inverted several times, for complete mixing of the sample with the reagents, and then placed on crushed ice. The tubes were allowed to stay on ice for at least 15 min, followed by centrifugation for about 15 min at 1300×g and 5° C. The tubes were then transported to a fume hood for transfer of the separated plasma to labeled screw-cap polypropylene tubes. Samples were then frozen and kept at −20° C. until assays were performed. A validated LC/MS/MS method was used for the analysis of unchanged omapatrilat in plasma. The plasma volume used for analysis was 0.5 mL. After the addition of internal standard and buffer solutions, the samples were extracted using ethyl acetate. The organic layer was separated and evaporated to dryness. The residue was then reconstituted and injected into the LC/MS/MS system. Chromatographic separation was achieved isocratically. Detection was by negative ion electrospray tandem mass spectrometry.

Pharmacokinetics:

Noncompartmental pharmacokinetic analysis was performed using published methods [Gibaldi M, Perrier D. In:Pharmacokinetics, 2^{nd} Edition. Marcel-Dekker, Inc., New York, N.Y., 1982, pp. 445-449.]

The results obtained are as follows.

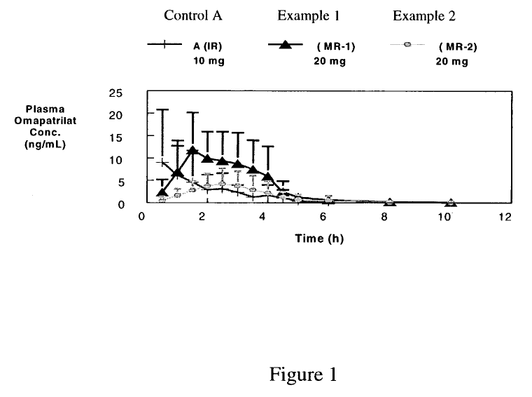

Plasma unchanged omapatrilat levels from the MR Examples 1 and 2 formulations and Control A IR formulation revealed differentiation within the first 4-6 h, after which the decline in concentrations was comparable for all treatments due to negligible colonic absorption (FIG. 1). Whereas the IR (Control A) formulation exhibited rapid attainment, of peak concentrations, both MR Examples 1 and 2 formulations exhibited more gradual rise in concentrations within the first couple of hours. The Example 1 MR formulation exhibited the following advantages over the IR formulation: Cmax of 20 mg Example 1 MR formulation was comparable to Cmax of 10 mg Control A IR, Tmax of Example 1 MR formulation was modestly prolonged compared to IR Control A, no loss in AUC (100% bioavailable relative to IR Control A dose), no risk of increased AUC due to dose dumping, relatively lower variability in Cmax compared to IR Control A dose, plasma omapatrilat levels modestly prolonged as compared to IR formulation. It is believed that these advantages translate to better efficacy at trough via improved plasma levels.

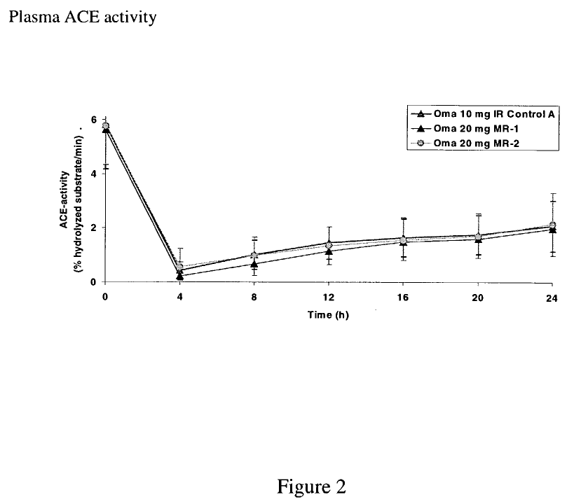

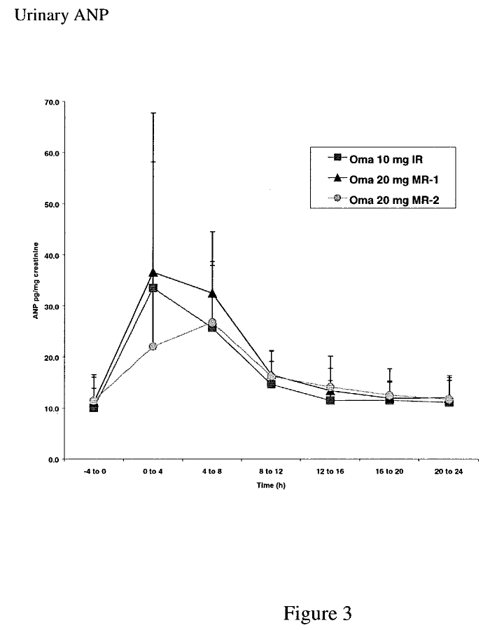

PD data (ACE, NEP inhibition) showed that both MR Examples 1 and 2 formulations behaved similarly, as compared to IR Control A indicating preservation of PD effects despite modifying release of the drug. In addition, the MR Examples 1 and 2 formulations demonstrated preserved ability to modulate BP, as shown by the 2 h post-dose BP measurements (Table 1). MR Example 2 formulation, while preserving ACE inhibition (FIG. 2), showed a delayed blunted peak effect on urinary ANP excretion (a marker of tissue NEP inhibition), compared to either MR Example 1 or IR Control A; this suggests an improved trough-to-peak ratio (reducing peak and maintaining trough) is feasible using a modified release approach (FIG. 3).

The Examples 1 and 2 MR formulations were comparable to the IR Control A formulation, in terms of tolerability and adverse events.

The above results are surprising since it would not be expected that the omapatrilat dose of 20 mg in MR Examples 1 and 2 of the invention would be tolerated as well as Control A IR tablets containing 10 mg omapatrilat and provide a Cmax comparable to the Control A IR tablets.

The Examples 1 and 2 omapatrilat MR formulations of the invention provided an overall substantially improved and more balanced drug release profile over the first few hours of release compared to that obtained with comparable (Control A) immediate-release tablets.

TABLE 1
Mean (±SD) Blood Pressure Response (mm Hg)

| | Omapatrilat | Omapatrilat 20 mg | Omapatrilat 20 mg |
|---|---|---|---|
| | 10 mg IR | MR Formulation | MR Formulation |
| | Control A | Example 1 | Example 2 |
| Mean SBP | 117 (10) | 117 (9) | 117 (9) |
| Pre-dose | | | |
| Mean SBP | 112 (10) | 114 (15) | 117 (13) |
| Post-dose | | | |
| Mean DBP | 68 (10) | 70 (8) | 72 (4) |
| Pre-dose | | | |
| Mean DBP | 59 (8) | 61 (10) | 64 (9) |
| Post-dose | | | |

### EXAMPLE 7

Experiment Comparing Modified-Release (MR) Formulations of the Invention (Examples 3, 4 and 5 and 30 mg Gemopatrilat Tablets) and Immediate-Release (IR) 30 mg Gemopatrilat Control B Tablets

A randomized, 2- or 3-way crossover study was performed in 20 or 24 healthy subjects to evaluate the oral pharmacokinetic (PK) and phamacodynamic (PD) performance of the Example 3 modified-release 30 mg gemopatrilat beadlets and the Examples 4 and 5 modified-release 30 mg gemopatrilat tablets.

Plasma samples were measured for unchanged gemopatrilat using a validated LC/MS/MS method, as described in Example 6, and PK characterized using a non-compartmental method, as described in Example 6.

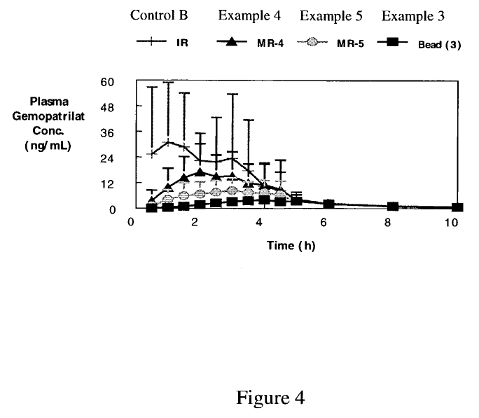

Plasma unchanged gemopatrilat levels from the Examples 3, 4 and 5 MR and IR Control B formulations (containing 30 mg gemopatrilat) revealed differentiation within the first 4-6 h (containing 30 mg gemopatrilat), after which the decline in concentrations was comparable for all treatments (FIG. 4). Whereas IR Control B formulation exhibited rapid attainment of peak concentrations, Examples 3, 4 and 5 MR formulations exhibited more gradual rise in concentrations within the first couple of hours. The desirable characteristics of the Example 4 MR formulation include: reduced Cmax compared to IR Control B, modest prolongation of Tmax compared to IR Control B, substantial preservation of AUC (66% bioavailable relative to IR), variability in Cmax for Example 4 MR comparable to IR Control B.

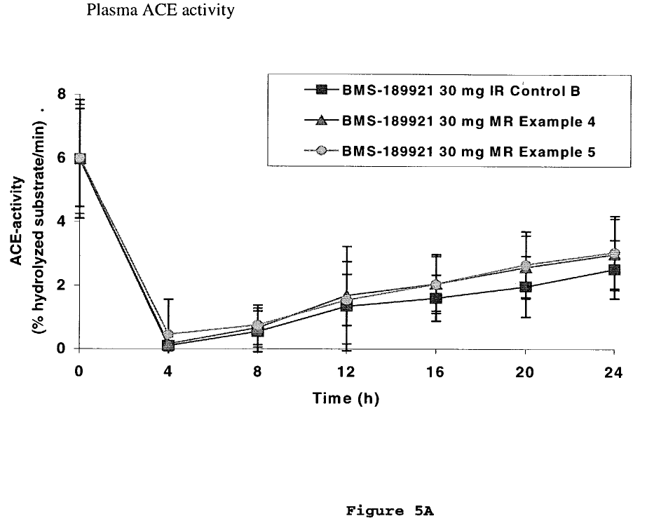

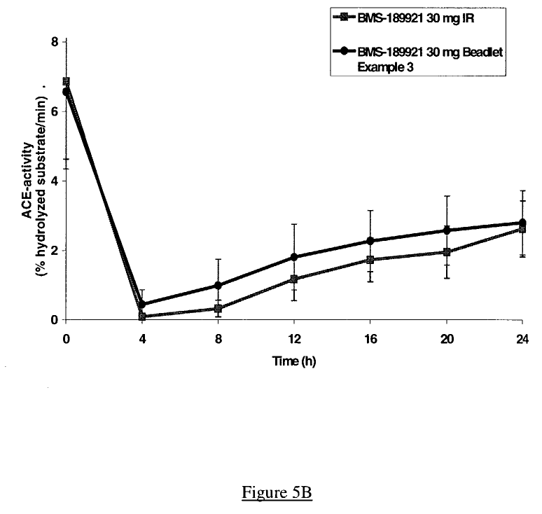

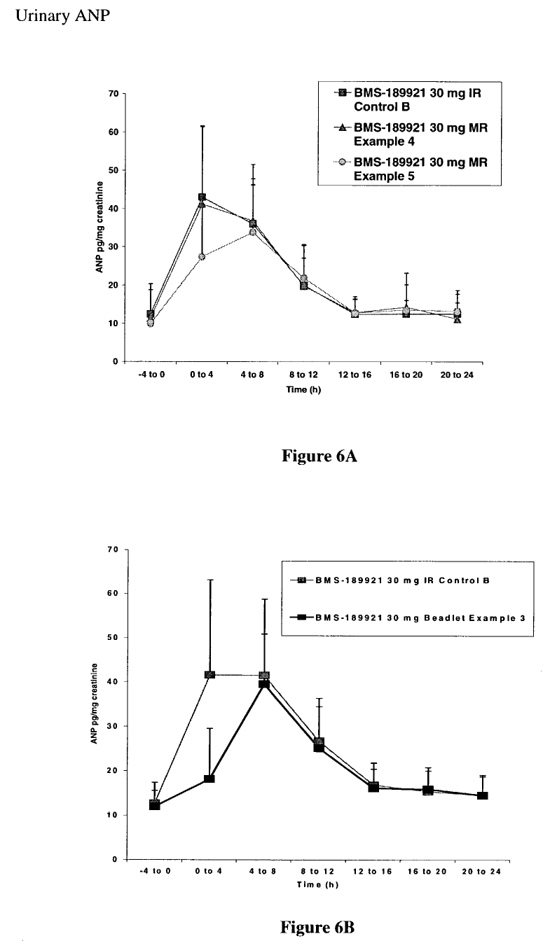

PD data (ACE, NEP inhibition) showed that all Examples 3, 4 and 5 MR formulations behaved similarly, as compared to IR Control B, indicating preservation of PD effects despite modifying release of the drug. In addition, Examples 3, 4 and 5 MR formulations demonstrated preserved ability to modulate BP, as shown by the 2 h post-dose BP measurements (Table 2). Example 5 MR tablets and Example 3 MR beadlets, while preserving ACE inhibition (FIG. 5), showed a delayed blunted peak effect on urinary ANP excretion (a marker of tissue NEP inhibition), compared to either Example 4 MR or IR Control B; this suggests an improved trough-to-peak ratio (reducing peak and maintaining trough) is feasible using a modified release approach (FIG. 6).

Examples 3, 4 and 5 gemopatrilat MR formulations of the invention were comparable to the IR Control B formulation in terms of tolerability and adverse events and provided an overall substantially improved and more balanced drug release profile over the first few hours of release compared to that obtained with comparable (Control B) immediate-release tablets.

TABLE 2
Gemopatrilat Formulations Mean (±SD)
Blood Pressure Response (mm Hg)

| | Gemopatrilat | Gemopatrilat 30 mg | Gemopatrilat 30 mg |
|---|---|---|---|
| | 30 mg IR | MR Formulation | MR Formulation |
| | Control B | Example 4 | Example 5 |
| Mean SBP | 118 (11) | 119 (14) | 116 (12) |
| Pre-dose | | | |
| Mean SBP | 110 14) | 109 (13) | 115 (13) |
| Post-dose | | | |
| Mean DBP | 69 (9) | 70 (11) | 69 (10) |
| Pre-dose | | | |
| Mean DBP | 58 (8) | 59 (8) | 61 (9) |
| Post-dose | | | |

Mean (±SD) Blood Pressure Response (mm Hg)

| | Gemopatrilat | Gemopatrilat 30 mg |
|---|---|---|
| | 30 mg IR | MR Beadlet |
| | (Control B) | Example 3 |
| Mean SBP Pre-dose | 115 (11) | 116 (12) |
| Mean SBP Post-dose | 108 (11) | 110 (10) |
| Mean DBP Pre-dose | 65 (10) | 65 (12) |
| Mean DBP Post-dose | 56 (10) | 61 (11) |

### EXAMPLE 8

Experiment Comparing Modified-Release (MR) Formulations of the Invention (Examples 1 and 2 20 mg Omapatrilat Tablets) and Immediate Release (IR 10 and 20 mg) Omapatrilat Control Tablets

A randomized, double blind, active-controlled trial was performed in 154 subjects with mild-to-moderate hypertension. After a three week placebo run-in, subjects were randomized to 8 weeks double-blind treatment (1 week lead-in dose/7 weeks target dose) with one of three dose regimens: omapatrilat 10 mg IR/20 mg MR-B (Example 1 formulation of the invention), omapatrilat 10 mg IR/20 mg MR-C (Example 2 formulation of the invention), or omapatrilat 10 mg IR/20 mg IR. All subjects received hydrochlorothiazide 25 mg open-label in addition to double-blind treatment during weeks 5-8. Ambulatory blood pressure was measured at baseline and Week 4. Trough blood pressure was measured at all visits.

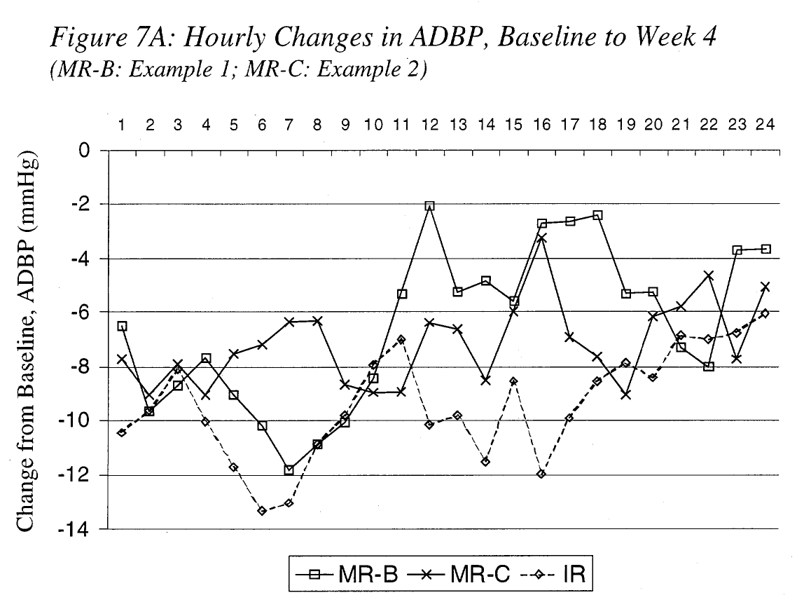

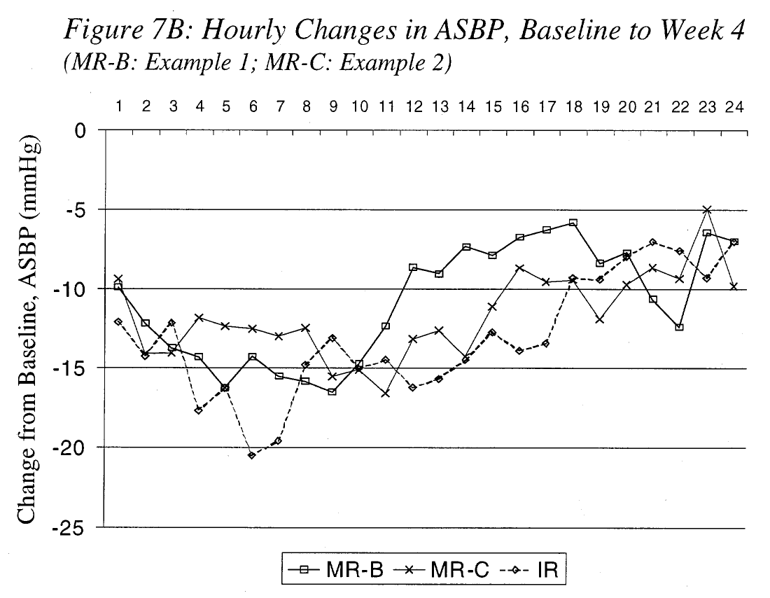

The ambulatory blood pressure data at Week 4 (hourly changes—FIG. 7A diastolic (ADBP) and FIG. 7B systolic (ASBP)) confirm that modified release can modulate the blood pressure vs. time profile. The marked peak in antihypertensive effect observed from hours 2 through 8 with the immediate release formulation is blunted with the modified release formulations, while the antihypertensive effect later in the dosing interval (hours 12 through 24) is preserved with both modified release formulations. Thus, the results of this study verify that improved pharmacodynamics, with smoother antihypertensive effect, and improved trough-to-peak ratio, is feasible using the modified release approach of the invention.

What is claimed is:

1. A modified-release pharmaceutical formulation comprising a pharmaceutical capable of providing both NEP inhibitory activity and ACE inhibitory activity, and a drug delivery system therefor which is capable of releasing said pharmaceutical at the desired site of absorption in a manner to provide therapeutically effective levels of NEP inhibitory activity and ACE inhibitory activity over a desired dosing interval.

2. The formulation as defined in claim 1 wherein the pharmaceutical is a vasopeptidase inhibitor.

3. The formulation as defined in claim 2 wherein the vasopeptidase inhibitor is omapatrilat or gemopatrilat.

4. The formulation as defined in claim 1 in the form of a modified-release coated formulation or a modified-release matrix formulation.

5. The formulation as defined in claim 4 in the form of a modified-release matrix formulation comprising (1) an inner solid particulate phase, and (2) an outer solid continuous phase in which particles of the inner solid particulate phase are dispersed and embedded, the particles of the inner solid particulate phase comprising (a) a vasopeptidase inhibitor; and (b) an optional extended release material, and the outer solid continuous phase comprising an extended release material.

6. The formulation as defined in claim 5 wherein the extended release material present in the inner solid particulate phase comprises one or more hydrophilic polymers, one or more hydrophobic polymers and/or one or more other type hydrophobic materials; and the extended release material in the outer solid continuous phase comprises one or more hydrophilic polymers, one or more hydrophobic polymers and/or one or more other type hydrophobic materials.

7. The formulation as defined in claim 5 wherein the inner solid particulate phase comprises omapatrilat or gemopatrilat, ethyl cellulose and/or sodium carboxymethyl cellulose and/or glycerylmonostearate, and the outer solid continuous phase comprises hydroxypropylmethylcellulose 2208 USP (100,000 cps), and/or hydroxypropylmethylcellulose 2910 USP (5 cps) and/or microcrystalline cellulose.

8. The formulation as defined in claim 4 wherein the modified-release coated formulation includes one or more modified-release coated cores comprising a vasopeptidase inhibitor and a modified-release coating therefor.

9. The formulation as defined in claim 2 which has the following release profile
C^{max} of from about 20 to about 80% of that obtained with a comparable immediate-release formulation
improved trough/peak ratio as compared to a comparable immediate-release formulation.

10. The formulation as defined in claim 1 which is designed to have reduced peak levels and improved trough/peak ratios as compared to a comparable immediate release formulation.

11. The formulation as defined in claim 1 wherein the drug delivery system is a diffusion controlled matrix system, an erodible/degradable matrix system, a dissolution controlled matrix system, an osmotic system or a barrier membrane system.

12. The formulation as defined in claim 2 which is designed to release vasopeptidase inhibitor at a rate to provide reduction in peak level and/or reduction in overall exposure of the vasopeptidase inhibitor to systemic circulation, and/or improvement in the NEP inhibition profile, improvement in the trough/peak ratio for blood pressure lowering, and/or reduction in dosing frequency and/or titrations, and/or improvement in patient compliance and/or improvement in tolerability, as compared to a comparable immediate or rapid release drug delivery system.

13. The formulation as defined in claim 1 which comprises
(a) core containing from about 1 to about 50% by weight (based on the weight of the total formulation) vasopeptidase inhibitor, and
(b) from about 99 to about 5% modified-release coating (based on the weight of the total formulation).

14. The formulation as defined in claim 13 in the form of modified-release coated beads which comprises
(a) core beads in an amount from about 50 to about 95% based on the weight of the modified-release coated bead;
(b) an optional subcoat in an amount from about 0 to about 35% based on the weight of the modified-release coated bead;
(c) a film coat or modified-release coat in an amount from about 65 to about 5% based on the weight of the modified-release coated bead; and
(d) an overcoat in an amount from about 0 to about 10% based on the weight of the modified-release coated bead.

15. The formulation as defined in claim 14 wherein the core bead comprises
(a) a vasopeptidase inhibitor which is omapatrilat or gemopatrilat
(b) one or more spheronizing aids and/or fillers which are selected from calcium phosphate (dibasic anhydrous and dibasic dihydrate), calcium sulfate, cellulose (powdered and silicified microcrystalline), dextrin/dextrates, lactose, maltidextrin, maltitol, mannitol, sorbitol, compressible sugars, xylitol, or any combination of the above listed excipients; and
wherein the optional subcoat comprises
(a) a hydrophilic polymer which is hydroxypropylmethyl cellulose, alginate, cellulose acetate, cellulose acetate phthalate, ethylcellulose, all grades of Methocel A, F, E, and K, hydroxypropyl derivatives of HPC, derivatives of HPMC, hydroxypropyl cellulose, hydroxypropyl methylcellulose phthalate, polymethacrylates, and xanthan gum and/or mixtures thereof, and
(b) a plasticizing agent which is polyethylene glycol, dibutyl sebacate, diethyl phthalate, glycerin, glyceryl monostearate, mineral oil and lanolin alcohols, petrolatum and lanolin alcohols, propylene glycol, triacetin, triethyl citrate and/or mixtures thereof;
and wherein the film coat or modified-release coat comprises
(a) one or more modified-release hydrophilic polymer which is one or more ammonio methacrylate copolymers (Eudragit RL 30D and/or RS 30D), carboxymethylcellulose sodium, cellulose acetate, cellulose acetate phthalate, ethylcellulose, hydroxyethyl cellulose, hydroxypropyl cellulose, hydroxypropyl methylcellulose, hydroxypropyl methylcellulose phthalate, methylcellulose, polymethacrylates, shellac, carnauba wax, microcrystalline wax, white wax, yellow wax, xanthan gum, zein and/or mixtures thereof;
(b) one or more plasticizing agents which is triethyl citrate, dibutyl sebacate, diethyl phthalate, glycerin, glyceryl monostearate, mineral oil and lanolin alcohols, petrolatum and lanolin alcohols, polyethylene gycol, propylene glycol, triacetin and/or mixtures thereof;
(c) optionally one or more anti-adherents which is magnesium silicate, talc, colloidal silicon dioxide, fumaric acid, glyceryl monostearate, glyceryl palmitostearate, isopropyl myristate, magnesium stearate, medium chain triglycerides, mineral oil, poloxamer, polyethylene glycol, polyoxyethylene stearates, sodium lauryl sulfate, sodium stearyl fumarate, stearic acid, titanium dioxide, vegetable oil, zinc stearate, and/or mixtures thereof;
(d) optionally one or more wetting agents which is polysorbate 80, docusate sodium, poloxamer, polyoxyethylenes (i.e. polyoxyethylene alkyl ethers, polyoxyethylene castor oil derivatives, polyoxyethylene sorbitan fatty acid esters, & polyoxyethylene stearates) sodium lauryl sulfate, and sorbitan esters (sorbitan fatty acid esters), and/or mixtures thereof; and
(e) optionally one or more antifoam agents which is simethicone; and
wherein the optional overcoat is comprised of
an anti-adherent which is magnesium silicate, talc, colloidal silicon dioxide, fumaric acid, glyceryl monostearate, glyceryl palmitostearate, isopropyl myristate, magnesium stearate, medium chain triglycerides, mineral oil, poloxamer, polyethyl glycol, polyoxyethylene stearates, sodium lauryl sulfate, sodium stearyl fumarate, stearic acid, titanium dioxide, vegetable oil, zinc stearate and/or mixtures thereof.

16. The composition as defined in claim 14 wherein said core is comprised of

| | % by weight * |
|---|---|
| Vasopeptidase inhibitor | 1 to 90 |
| Filler | 10 to 99 |

(* % based on core weight)
said modified-release coating is comprised of

| | |
|---|---|
| methacrylic | Range (% by weight**) |
| acid copolymers | 20 to 70% |
| plasticizer | 2 to 40% |

(**% based on weight of film coat).

17. The formulation as defined in claim 13 comprising 20 mg omapatrilat coated at 25% modified-release coating level or 30 mg gemopatrilat coated at 10% or less modified-release coating level.

18. The formulation as defined in claim 1 in the form of modified-release tablets of the following formulations:

| Formulation 1 | | Formulation 2 | |
|---|---|---|---|
| Ingredient | Mg/Tablet | Ingredient | Mg/Tablet |
| Omapatrilat | 20.0 | Omapatrilat | 20.0 |
| Anhydrous Lactose | 93.0 | Anhydrous Lactose | 83.0 |
| Microcrystalline | 30.0 | Microcrystalline | 30.0 |
| Cellulose | | Cellulose | |
| Hydroxypropyl | 30.0 | Hydroxypropyl | 60.0 |
| methylcellulose | | methylcellulose | |
| Hydroxypropyl | 20.0 | Stearic Acid | 6.0 |
| methylcellulose | | | |
| Stearic Acid | 6.0 | Silicon Dioxide | 1.0 |
| Silicon Dioxide | 1.0 | | |
| Tablet Weight | 200.0 | Total Weight | 200.0 |

19. The formulation as defined in claim 16 in the form of a capsule containing modified-release beadlets having the following formulation:

| Ingredient | Mg/Capsule |
|---|---|
| Core Beads | |
| Gemopatrilat | 30.0 |
| Microcrystalline Cellulose | 90.0 |
| Seal Coat | 3.6 |
| Hydroxypropyl cellulose | |
| Modified-Release Coat | |
| Polymethacrylic acid esters | 7.0 |
| Triethyl Citrate | 1.2 |
| Talc | 6.0 |
| Encapsulation | |
| Beadlets | 137.8 |
| Hard Gelatin Capsule | 1 capsule |

20. The formulation as defined in claim 2 in the form of a tablet having the following formulation:
Formulation A

| Ingredient | Mg/Tablet |
|---|---|
| Gemopatrilat | 30.0 |
| Anhydrous Lactose | 124.5 |
| Microcrystalline Cellulose | 45.0 |
| Hydroxypropyl Methyl Cellulose | 90.0 |
| Stearic Acid | 9.0 |
| Silicon Dioxide | 1.5 |
| Tablet Weight | 300.0 or |

Formulation B

| Ingredient | Mg/Tablet |
|---|---|
| Gemopatrilat | 30.0 |
| Anhydrous Lactose | 139.5 |
| Microcrystalline Cellulose | 45.0 |
| Hydroxypropyl methylcellulose | 45.0 |
| (Methocel K100LV) | |
| Hydroxypropyl methylcellulose | 30.0 |
| (Methocel K4M) | |
| Stearic Acid | 9.0 |
| Silicon Dioxide | 1.5 |
| Tablet Weight | 300.0 |

21. A method for the lowering of blood pressure and/or treating renal diseases in a human or animal in need thereof, which comprises administering in a time-controlled manner a therapeutically effective amount of a pharmaceutical formulation as defined in claim 1 which provides therapeutically effective amounts of both ACE inhibitory activity and NEP inhibitory activity, to effect lowering of blood pressure and/or treat renal diseases over a desired period.

22. The method as defined in claim 21 wherein said pharmaceutical is a vasopeptidase inhibitor which is omapatrilat or gemopatrilat, CGS 30440 or MD 100240.

23. The method as defined in claim 22 wherein the NEP inhibitory activity is modulated while the ACE inhibitory activity is substantially unaffected as compared to a comparable immediate- or rapid-release drug delivery system.

24. The method as defined in claim 23 wherein the modified-release of the vasopeptidase inhibitor provides a reduction in peak level and/or reduction in overall exposure of the vasopeptidase inhibition profile, and/or improvement in the trough/peak ratio for blood pressure lowering, and/or reduction in dosing frequency and/or titrations, and/or improvement in patient compliance and/or improvement in tolerability as compared to a comparable immediate- or rapid-release drug delivery system.

25. The method as defined in claim 22 wherein the duration of NEP inhibitory activity profile is balanced to more closely resemble or be more closely aligned with the ACE inhibitory activity profile, while minimally affecting the ACE inhibitory activity.

26. The method as defined in claim 21 wherein the vasopeptidase inhibitor is continuously introduced into the environment of use over a period from about 4 to about 24 hours.

27. The method as defined in claim 21 wherein the pharmaceutical is a combination of a vasopeptidase inhibitor and a diuretic.

28. The method as defined in claim 27 wherein the pharmaceutical is omapatrilat and hydrochlorothiazide or furosemide.

29. A method for improving drug release properties of a formulation containing a vasopeptidase inhibitor, which comprises formulating said vasopeptidase inhibitor in a modified-release formulation capable of releasing therapeutically effective amounts of vasopeptidase in a patient in a more balanced manner over the first few hours of release as compared to comparable immediate-release formulations.

30. A method for improving NEP inhibitory activity profile of a vasopeptidase inhibitor, which comprises formulating said vasopeptidase inhibitor in a modified-release formulation capable of releasing therapeutically effective amounts of vasopeptidase inhibitor in a patient in a more balanced manner over the first few hours of release as compared to comparable immediate-release formulations.

31. The method as defined in claim 30 wherein the modified-release formulation provides a C^{max} of from about 20 to about 80% of that obtained with a comparable immediate release formulation, and an improved peak to trough ratio as compared to comparable immediate-release formulations.

32. A pharmaceutical combination comprising the formulation as defined in claim 1 and one or more other therapeutic agents.

33. The pharmaceutical combination as defined in claim 32 wherein the other therapeutic agent is selected from one or more diuretics, one or more antihypertensive agents, one or more platelet aggregation inhibitors, one or more other cardiovascular agents, one or more anti-anginal agents, one or more anti-arrhythmic agents, one or more anti-atherosclerosis agents, one or more hypolipidemic agents lipid-lowering agents, lipid agents, or lipid modulating agents, one or more other antidiabetic agents, anti-obesity agents, anti-dementia agents, anti-Alzheimer's agents, anti-osteoporosis agents, hormone replacement therapeutic agents, anti-inflammatory agents, anti-arthritis agents, anti-platelet agents, anti-heart failure agent), anti-cancer agents, anti-infective agents, growth hormone secretagogues, selective androgen receptor modulators, and/or immunomodulatory agents.

34. The combination as defined in claim 33 wherein the formulation includes a diuretic in the same or different dosage form.

35. The combination as defined in claim 34 wherein the diuretic is hydrochlorothiazide, torasemide, furosemide, dichlorophenamide, spironolactone, indapamide, polythiazide, methclothiazide, chlorothiazide, hydroflumethiazide, ethacrynic acid, triamterene, amiloride, metolazone, chlorthalidone, and mixtures of two or more thereof.

36. The combination as defined in claim 34 comprising said formulation containing omapatrilat or gemopatrilat and as the diuretic hydrochlorothiazide or furosemide.

37. The combination as defined in claim 33 comprising a combination of immediate release hydrochlorothiazide coating a matrix core comprising sustained release omapatrilat.

38. The combination as defined in claim 33 wherein the antihypertensive agent employed is an ACE inhibitor, angiotensin II receptor antagonist, NEP inhibitor, a NEP/ACE inhibitor, a calcium channel blocker, a T-channel calcium antagonist, a β-adrenergic blocker, a diuretic, a (-adrenergic blocker, a dual action receptor antagonist (DARA), or a heart failure drug, the hypolipidemic agent or lipid-lowering agent or other lipid agent or lipid modulating agent or anti-atherosclerotic agent, which is employed comprises 1,2,3 or more MTP inhibitors, HMG CoA reductase inhibitors, squalene synthetase inhibitors, fibric acid derivatives, PPAR α agonists, PPAR dual α/γ agonists, PPAR δ agonists or antagonists, ACAT inhibitors, lipoxygenase inhibitors, cholesterol absorption inhibitors, ileal Na⁺/bile acid cotransporter inhibitors, upregulators of LDL receptor activity, cholesteryl ester transfer protein inhibitors, bile acid sequestrants, or nicotinic acid and derivatives thereof, ATP citrate lyase inhibitors, phytoestrogen compounds, an HDL upregulators, LDL catabolism promoters, antioxidants, PLA-2 inhibitors, antihomocysteine agents, HMG-COA synthase inhibitors, lanosterol demethylase inhibitors, or sterol regulating element binding protein-I agents; and the antidiabetic agent which may be optionally employed is 1,2,3 or more insulin secretagogues or insulin sensitizers, biguanides, sulfonyl ureas, PTP-1B inhibitors, aldose reductase inhibitors, glucosidase inhibitors, PPAR γ agonists, PPAR α agonists, PPAR δ antagonists or agonists, aP2 inhibitors, PPAR α/γ dual agonists, dipeptidyl peptidase IV (DP4) inhibitors, SGLT2 inhibitors, glycogen phosphorylase inhibitors, and/or meglitinides, insulin, and/or glucagon-like peptide-1 (GLP-1) or mimetics thereof, or the other type of therapeutic agent which may be optionally employed is 1, 2, 3 or more of an anti-obesity agent which is a beta 3 adrenergic agonist, a lipase inhibitor, a serotonin (and dopamine) reuptake inhibitor, an aP2 inhibitor, a thyroid receptor beta drug, an anorectic agent, a PTP-1B inhibitor, a CCKA agonist, a neuropeptide Y antagonist, a melanocortin-4-receptor agonist, a PPAR modulator which is a PPAR γ antagonist, PPAR a agonist, and/or PPAR 6 antagonist, a leptin inhibitor which is a leptin receptor activator, or a fatty acid oxidation upregulator or inducer, the lipid modulating agent is an MTP inhibitor, an HMG CoA reductase inhibitor, a squalene synthetase inhibitor, a fibric acid derivative, an upregulator of LDL receptor activity, a lipoxygenase inhibitor, or an ACAT inhibitor and the other lipid agent is a cholesteryl ester transfer protein inhibitor; the other therapeutic agent is an anti-Alzheimer's agent or anti-dementia agent, which is tacrine HCl, donepezil, a Υ-secretase inhibitor, a β-secretase inhibitor and/or antihypertensive agent;
an antiosteoporosis agent, which is parathyroid hormone, a bisphosphonate, alendronate, a Ca receptor agonist or a progestin receptor agonist;
a hormone replacement therapeutic agent, which is a selective estrogen receptor modulator (SERM);
a tyrosine kinase inhibitor;
a selective androgen receptor modulator;
an antiarrhythmic agent, which is a β-blocker, or a calcium channel blocker, or an α-adrenergic blocker;
coenzyme Q sub. 10;
an agent that upregulates type III endothelial cell nitric acid syntase;
a chondroprotective compound which is polysulfated glycosaminoglycan (PSGAG), glucosamine, chondroitin sulfate (CS), hyaluronic acid (HA), pentosan polysulfate (PPS), doxycycline or minocycline;
a cyclooxygenase (COX)-2 inhibitor, which is Celebrex® or Vioxx® or a glycoprotein IIa/IIIb receptor antagonist;
a 5-HT reuptake inhibitor;
a growth hormone secretagogue;
an anti-atherosclerosis agent;
an anti-infective agent, or an immunosuppressant for use in transplantation, or an antineoplastic agent, the antihypertensive agent is an ACE inhibitor which is captopril, fosinopril, enalapril, lisinopril, quinapril, benazepril, fentiapril, ramipril or moexipril;
an angiotensin II receptor antagonist which is irbesartan, losartan or valsartan;
amlodipine besylate, prazosin HCl, verapamil, nifedipine, nadolol, propranolol, or clonidine HCl, carvediol, atenolol, hydrochlorothiazide, torasemide, furosemide, spironolactone or indapamide, and the platelet aggregate inhibitor is clopidogrel, aspirin or a combination of clopidogrel and aspirin, the antidiabetic agent is 1, 2, 3 or more of metformin, glyburide, glimepiride, glipyride, glipizide, chlorpropamide, gliclazide, acarbose, miglitol, pioglitazone, troglitazone, rosiglitazone, insulin, Gl-262570, isaglitazone, JTT-501, NN-2344, L895645, YM-440, R-119702, AJ9677, repaglinide, nateglinide, KAD1129, AR-HO39242, GW-409544, KRP297, AC2993, LY315902, P32/98 and/or NVP-DPP-728A; the anti-obesity agent is orlistat, ATL-962, AJ9677, L750355, CP331648, sibutramine, topiramate, axokine, dexamphetamine, phentermine, phenylpropanolamine, and/or mazindol, P57 or CP-644673 (Pfizer), the lipid modulating agent is pravastatin, lovastatin, simvastatin, atorvastatin, cerivastatin, fluvastatin, pitavastatin, rosuvastatin, fenofibrate, gemfibrozil, clofibrate, avasimibe, TS-962, MD-700, cholestagel, niacin, and/or LY295427.
